(19) United States
(12) Patent Application Publication
(10) Pub. No.: US 2025/0170072 A1
(43) Pub. Date: May 29, 2025

(54) NOVEL COMPOSITIONS FOR REDUCING METHANE PRODUCTION IN RUMINANT ANIMALS

(71) Applicant: FUTUREFEED PTY LTD, Brisbane, Queensland (AU)

(72) Inventors: Robert Douglas KINLEY, Wulguru, Queensland (AU); Breanna Michelle ROQUE, Garbutt, Queensland (AU); Siong TAN, Brooklyn, Victoria (AU); Jessica Ellen HARRIS, Oonoonba, Queensland (AU)

(73) Assignee: FUTUREFEED PTY LTD, Brisbane, Queensland (AU)

(21) Appl. No.: 18/837,062

(22) PCT Filed: Feb. 9, 2023
(86) PCT No.: PCT/AU2023/050085

§ 371 Date: Aug. 8, 2024

(30) Foreign Application Priority Data
Feb. 9, 2022 (AU) 2022900266

(51) Int. Cl. A61K 31/02; A23K 20/105; A23K 20/158; A23K 20/163; A23K 50/10; A61K 47/40; A61K 47/44; A61K 47/46

(52) U.S. Cl. CPC A61K 31/02; A23K 20/105; A23K 20/158; A23K 20/163; A23K 50/10; A61K 47/40; A61K 47/44; A61K 47/46

(57) ABSTRACT

The present invention relates to processes for preparing compositions suitable for reducing total gas production and/or methane production and/or growth performance in a ruminant animal.

### FIELD OF THE INVENTION

The field of the invention relates to processes for preparing compositions suitable for reducing total gas production and/or methane production and/or growth performance in a ruminant animal.

### BACKGROUND OF THE INVENTION

Methane (CH₄) is a greenhouse gas (GHG) produced primarily by methanogenic microbes that are found in natural ecosystems (e.g. wetlands, oceans and lakes) and the gastrointestinal tract of invertebrates and vertebrates, such as termites and ruminants. Every year ˜429-507 Tg of CH₄ are removed from the atmosphere and ˜40 Tg from the stratosphere through reactions with hydroxyl (OH) radicals; and ˜30 Tg by CH₄-oxidizing bacteria in soil.

Nevertheless, anthropogenic GHG emissions have been increasing rapidly, with the CH₄ concentration in the atmosphere now more than twofold higher than in the early 1800s. Methane is very effective in absorbing solar infrared radiation and has a global warming potential 28 times greater than CO₂. Consequently, its accumulation in the atmosphere contributes considerably to climate change. One of the main sources of anthropogenic CH₄ can be attributed to agricultural activities, including ruminant livestock.

According to a recent UN report, cattle-rearing generates more global warming greenhouse gases, as measured in CO₂ equivalent, than transportation. In Australia, ruminants are estimated to contribute ˜10% of the total GHG emissions. Ruminants produce CH₄ as a by-product of the anaerobic microbial fermentation of feeds in the rumen and, to a lesser extent, in the large intestine. The ruminal microbial community is highly diverse and composed of bacteria, protozoa, fungi, and bacteriophages that act collectively to ferment ingested organic matter (OM), resulting in CO₂, H₂, volatile fatty acids (VFAs), and formates. Methanogenic archaea present in the rumen use these end-products and produce CH₄. Although the production of CH₄ reduces the partial pressure of H₂, which could otherwise inhibit rumen fermentation, it also reduces the amount of energy and carbon available for formation of VFAs essential for ruminant nutrition. Most of the CH₄ produced in ruminants is exhaled and belched by the animal and represents a loss of up to 12% of gross energy intake.

Previous work has focused on the use of Asparagopsis species as feed supplements for reducing total gas production and/or methane production in ruminant animals. This work has led to the use of Asparagopsis biomass and Asparagopsis derived products for reducing methane, but there remains a need for further mitigation strategies that reduce enteric CH₄ formation.

### SUMMARY OF THE INVENTION

In one aspect, the present invention provides a method for reducing total gas production and/or methane production in a ruminant animal comprising the step of providing said ruminant animal with an effective amount of a composition comprising manufactured bromoform and a bromoform stabilising excipient.

In one embodiment, the step of providing said ruminant animal an effective amount of a composition comprises administering said ruminant animal with the effective amount of a composition comprising manufactured bromoform in a bromoform stabilising excipient.

In another embodiment, the step of providing said ruminant animal an effective amount of a composition comprises making the composition comprising manufactured bromoform in a bromoform stabilising excipient available in a feed system comprising the ruminant animal.

In another embodiment, the composition is administered at a dose of at least 0.005, 0.01, 0.02, 0.03, 0.04, 0.05, 0.06 or 0.08 mg of bromoform per gram of organic matter provided to the ruminant animal.

In another embodiment, the composition is made available at an amount to provide a dose of at least 0.005, 0.01, 0.02, 0.03, 0.04, 0.05, 0.06 or 0.08 mg of bromoform per gram of organic matter provided to the ruminant animal.

In another embodiment, said ruminant animal is selected from the members of the Ruminantia and Tylopoda suborders.

In another embodiment, said ruminant animal is cattle or sheep.

In another embodiment, the bromoform stabilising excipient is an edible non-polar substance.

In another embodiment, the edible non-polar substance is an edible oil.

In another embodiment, the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In another embodiment, the bromoform stabilising excipient is an edible carbohydrate, or water.

In another embodiment, the carbohydrate is a cyclodextrin or a molasses.

In another embodiment, the composition is in a solid, a semi-solid or a liquid form.

In another aspect, the present invention provides a composition when used for reducing total gas production and/or methane production in a ruminant animal, wherein said composition comprises manufactured bromoform and a bromoform stabilising excipient.

In one embodiment the present invention provides a composition when used as described herein, wherein the bromoform stabilising excipient is an edible non-polar substance.

In one embodiment the present invention provides a composition when used as described herein, wherein the edible non-polar substance is an edible oil.

In one embodiment the present invention provides a composition when used as described herein, wherein the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In one embodiment the present invention provides a composition when used as described herein, wherein the bromoform stabilising excipient is an edible carbohydrate, or water.

In one embodiment the present invention provides a composition when used as described herein, wherein the carbohydrate is a cyclodextrin or a molasses.

In one embodiment the present invention provides a composition when used as described herein, wherein the composition formulated for provision to the ruminant animal at a dose of at least 0.005, 0.01, 0.02, 0.03, 0.04, 0.05, 0.06 or 0.08 mg of bromoform per gram of organic matter provided to the ruminant animal.

In one embodiment the present invention provides a composition when used as described herein, wherein the composition is in a solid, a semi-solid or a liquid form.

In one aspect, the present invention provides a feed supplement when used for reducing total gas production and/or methane production in a ruminant animal, said supplement comprising an effective amount of a composition comprising manufactured bromoform and a bromoform stabilising excipient.

In one embodiment the present invention provides a feed supplement when used as described herein, wherein the feed supplement is formulated to provide a dose of at least 0.005, 0.01, 0.02, 0.03, 0.04, 0.05, 0.06 or 0.08 mg of bromoform per gram of organic matter provided to the ruminant animal.

In another embodiment the present invention provides a feed supplement when used as described herein, wherein the supplement further comprises one or more edible excipients.

In a further embodiment the present invention provides a feed supplement when used as described herein, wherein said ruminant animal is selected from the members of the Ruminantia and Tylopoda suborders.

In a further embodiment the present invention provides a feed supplement when used as described herein, wherein said ruminant animal is cattle or sheep.

In a further embodiment the present invention provides a feed supplement when used as described herein, wherein the bromoform stabilising excipient is an edible non-polar substance.

In a further embodiment the present invention provides a feed supplement when used as described herein, wherein the edible non-polar substance is an edible oil.

In a further embodiment the present invention provides a feed supplement when used as described herein, wherein the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In a further embodiment the present invention provides a feed supplement when used as described herein, wherein the bromoform stabilising excipient is an edible carbohydrate, or water.

In a further embodiment the present invention provides a feed supplement when used as described herein, wherein the carbohydrate is a cyclodextrin or a molasses.

In a further embodiment the present invention provides a feed supplement when used as described herein, wherein the composition is in a solid, a semi-solid or a liquid form.

In one aspect, the present invention provides a method of producing a methane reducing ruminant animal feed, comprising mixing a ruminant animal feed with a feed supplement comprising an effective amount of a composition comprising manufactured bromoform and a bromoform stabilising excipient.

In one embodiment the present invention provides a method as described herein, wherein the animal feed comprises a dose of at least 0.005, 0.01, 0.02, 0.03, 0.04, 0.05, 0.06 or 0.08 mg of bromoform per gram of organic matter of the ruminant animal feed.

In one aspect, the present invention provides an animal feed when used for reducing total gas production and/or methane production in a ruminant animal, said animal feed comprising an effective amount of a composition comprising manufactured bromoform and a bromoform stabilising excipient and a ruminant animal feed.

In one embodiment, the present invention provides an animal feed when used as described herein, wherein the composition is formulated to provide a dose of at least 0.005, 0.01, 0.02, 0.03, 0.04, 0.05, 0.06 or 0.08 mg of bromoform per gram of the organic matter of the ruminant animal feed.

In one embodiment, the present invention provides an animal feed when used as described herein, wherein said ruminant animal is selected from the members of the Ruminantia and Tylopoda suborders.

In another embodiment, the present invention provides an animal feed when used as described herein, wherein said ruminant animal is cattle or sheep.

In a further embodiment, the present invention provides an animal feed when used as described herein, wherein the bromoform stabilising excipient is an edible non-polar substance.

In a further embodiment, the present invention provides an animal feed when used as described herein, wherein the edible non-polar substance is an edible oil.

In a further embodiment, the present invention provides an animal feed when used as described herein, wherein the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In a further embodiment, the present invention provides an animal feed when used wherein the bromoform stabilising excipient is an edible carbohydrate, or water.

In a further embodiment, the present invention provides an animal feed when used wherein the carbohydrate is a cyclodextrin or a molasses.

In a further embodiment, the present invention provides an animal feed when used as described herein, wherein the composition is in a solid, a semi-solid or a liquid form.

In one embodiment, the present invention provides a stabilised bromoform composition comprising manufactured bromoform and a bromoform stabilising excipient, wherein the bromoform stabilising excipient is selected from the group consisting of an edible solid, edible semi solid or edible liquid.

In one embodiment, the present invention provides a stabilised bromoform composition as described herein, wherein the bromoform stabilising excipient is selected from the group consisting of an edible oil, an edible carbohydrate and water.

In one embodiment, the present invention provides a stabilised bromoform composition as described herein, wherein the composition does not comprise one or more compounds selected from the group consisting of iodine, bromine, dibromochloromethane, bromochloroacetic acid, and dibromoacetic acid.

In one embodiment, the present invention provides a method of making a stabilised bromoform composition comprising contacting manufactured bromoform and a bromoform stabilising excipient, wherein the bromoform stabilising excipient is selected from the group consisting of an edible solid, edible semi solid or edible liquid.

In one embodiment, the present invention provides a method of making a stabilised bromoform composition as described herein, wherein the bromoform stabilising excipient is selected from the group consisting of an edible oil, an edible carbohydrate and water.

In one embodiment, the present invention provides a method of making a stabilised bromoform composition, wherein the composition does not comprise one or more compounds selected from the group consisting of iodine, bromine, dibromochloromethane, bromochloroacetic acid, and dibromoacetic acid.

### BRIEF DESCRIPTION OF THE DRAWINGS

FIG. 1 shows data in relation to effect of inclusion of an Asparagopsis-derived composition comprising Asparagopsis biomass extracted into oil (Asp-Oil) and a composition comprising manufactured bromoform in oil (Syn-Oil) on Methane, Hydrogen, Carbon Dioxide and Total Gas Production (mL/g fed) in vitro. Inclusion rates are based on bromoform concentrations of 0.08-0.26 mg. Gas results are presented on a mL per gram of Rhodes grass substrate fed because digestibility was not measured in Experiment 1. No standard errors are shown (experiment performed once).

FIG. 2 shows the effect of Low (0.04 mg), Medium (0.07 mg), and High (0.11 mg) inclusion of an Asparagopsis-derived composition comprising Asparagopsis biomass extracted into oil (Asp-Oil) and a composition comprising manufactured bromoform in oil (Syn-Oil) on Methane, Hydrogen, Total Gas Production, and in vitro dry matter digestibility (IVDMD). Gas results are presented on a mL per gram of dry matter digestibility (mL/g IVDDM. No standard errors are presented (experiment performed once).

FIG. 3 shows methane (CH₄) produced for Control (no bromoform; CHBr₃), canola oil containing manufactured synthetic CHBr₃ [“Canola Oil”], naturally derived CHBr₃ from Asparagopsis [“Asp-Oil”] and freeze-dried Asparagopsis [“FD-Asp”] at varying levels of CHBr₃ inclusion [0.03, 0.05, & 0.08 mg CHBr₃ per g feed substrate] during 24, 48, and 72 hours of in vitro rumen fermentation. Values expressed as mL per g digested dry matter (IVDDM). Lower case letters signify statistical significance at P<0.05 within each period.

FIG. 4 shows hydrogen (H₂) produced for Control (no bromoform; CHBr₃), canola oil containing manufactured synthetic CHBr₃ [Canola Oil], naturally derived CHBr₃ from Asparagopsis [Asp-Oil] and freeze-dried Asparagopsis [FD-Asp] at varying levels of CHBr₃ inclusion [0.03, 0.05, & 0.08 mg CHBr₃ per g feed substrate] during 24, 48, and 72 hours of in vitro rumen fermentation. Values expressed as mL per g digested dry matter (IVDDM). Lower cases letters signify statistical significance at P<0.05 within each period whereas ns signifies not significant.

FIG. 5 shows in vitro digested dry matter (IVDMD) [A], total volatile fatty acid production (tVFA) presented in millimolar (mM) [B], Acetate, Propionate, Butyrate presented as a percentage of tVFA, [C] and the ratio of Acetate to Propionate (A:P) [D] produced for Control (no bromoform; CHBr₃), canola oil containing manufactured synthetic CHBr₃ [Canola Oil], naturally derived CHBr₃ from Asparagopsis [Asp-Oil] and freeze-dried Asparagopsis [FD-Asp] at varying levels of CHBr₃ inclusion [0.03, 0.05, & 0.08 mg CHBr₃ per g feed substrate] during 72 hours of in vitro rumen fermentation. Lower cases letters signify statistical significance at P<0.05 within each period whereas ns signifies not significant.

FIG. 6 shows methane (CH₄) produced for Control (no bromoform; CHBr₃), and the four CHBr₃ stabilizing excipients [Corn Oil, Cyclodextrin, Molasses, and Water] at varying levels of CHBr₃ inclusion [0.01, 0.04, & 0.07 mg CHBr₃ per g feed substrate] during 24, 48, and 72 hours of in vitro rumen fermentation. Values expressed as mL per g digested dry matter (IVDDM). Lower cases letters signify statistical significance at P<0.05 for each period.

FIG. 7 shows hydrogen (H₂) produced for Control (no bromoform; CHBr₃), and four CHBr₃ stabilizing excipients [Corn Oil, Cyclodextrin, & Molasses] at varying levels of CHBr₃ inclusion [0.01, 0.04, & 0.07 mg CHBr₃ per g feed substrate] during 24, 48, and 72 hours of in vitro rumen fermentation. Values expressed as mL per g digested dry matter (IVDDM). Lower cases letters signify statistical significance at P<0.05 for each period.

FIG. 8 shows in vitro digested dry matter (IVDMD) [A], total volatile fatty acid production (tVFA) presented in millimolar (mM) [B], Acetate, Propionate, Butyrate presented as a percentage of tVFA, [C] and the ratio of Acetate to Propionate (A:P) [D] produced for Control (no bromoform; CHBr3) and four CHBr3 stabilizing excipients [Corn Oil, Cyclodextrin, & Molasses] at varying levels of CHBr3 inclusion [0.01, 0.04, & 0.07 mg CHBr3 per g feed substrate] during 72 hours of in vitro rumen fermentation. Lower cases letters signify statistical significance at P<0.05 for each period.

FIG. 9 shows percent methane (CH₄) reductions for stabilising excipients with manufactured synthetic bromoform (CHBr₃) [Grey Bars; Corn Oil, Cyclodextrin, Molasses, Canola Oil, & Water] and two Asparagopsis products [Black Bars] either freeze-dried [FD-Asp] or in canola oil [Asp-Oil] containing natural CHBr₃ CHBr₃ at varying levels of CHBr₃ inclusion [0.01-0.08 mg CHBr₃ per g feed substrate] during 24 (#1), 48 (#2), and 72 (#3) hours of in vitro rumen fermentation.

FIG. 10 shows methane (CH4) Production (grams CH4/day) and CH4 Yield (grams CH4/kg DMI) reduction expressed as a percentage for manufactured synthetic bromoform (CHBr3) stabilized in canola oil [Canola Oil] and naturally derived CHBr3 stabilized in canola oil [Asp-Oil] at 4 inclusion levels; 6, 12, 18, and 20 mg CHBr3/kg dry matter intake when fed to lactating dairy cattle in a pulse feeding system.

FIG. 11 shows bromoform (CHBr₃) content (mg/g) after 1, 2, 4 weeks of storage at 40° C., 25° C., 4° C. and −20° C. in airtight conditions and 40° C. and 25° C. in open air conditions in A) Corn Oil, B) Molasses and C) Cyclodextrin. Values are means ±SE (n=3).

### DETAILED DESCRIPTION

As will be discussed below, the present invention is based in part on the characterisation of compositions that are able to inhibit methane production by ruminal fermentation using manufactured bromoform. The compositions have anti-methanogenic activity at doses of manufactured bromoform demonstrated previously—in different compositions—to not significantly inhibit methane production. The development of compositions techniques to deliver Asparagopsis products into diets of livestock on rangelands allows for maximal distribution and/or environmental benefits, particularly in countries where the red meat, dairy, and wool industries, by animal head count, are largely a pastoral industry.

The present invention provides stabilised compositions comprising manufactured bromoform. The ability to prepare stabilised compositions comprising manufactured bromoform allows for the use of low doses of manufactured bromoform to inhibit methane production from ruminant animals, including at doses of bromoform demonstrated previously to not inhibit methane production.

Because low doses of manufactured bromoform can be used, bioavailable formulations can be delivered to animals in farming systems, such as pasture systems.

The ability to prepare stabilised compositions comprising manufactured bromoform also allows for the preparation of compositions of known sustained release characteristics, including at different temperatures.

The compositions described herein allow high levels of undesired trace elements and minerals to be avoided.

In vitro studies using rumen fluid have demonstrated that the production of CH₄ is significantly affected by synthetic halomethanes when these synthetic compounds are added to rumen fluid. For example, bromoform and dibromochloromethane in dimethyl sulfoxide (DMSO), when added to rumen fluid significantly inhibit the production of CH₄ compared to control when added to rumen fluid at concentrations ≥5 μM (Machado, L., Magnusson, M., Paul, N. A. et al. J Appl Phycol (2016) 28:3117).

However, previous work has demonstrated that the effects of Asparagopsis biomass containing secondary metabolites—or Asparagopsis derived products—both in rumen fluid and in vivo are variable and contradictory due to the differences in extracts/compositions comprising such compounds, doses of compounds, and the influence type and quality of basal diet.

Bromoform—one of the secondary metabolites contained in Asparagopsis biomass—is volatile and has physical properties (including its volatility) which are considered to make impractical its use in vivo or in vitro. Initial work demonstrated that manufactured bromoform in DMSO is unable to significantly inhibit methane production at low doses (e.g. doses of less than 5 μM) (Machado et al. (2016) J Appl Phycol 28:3117-3126). Subsequent work by Machado et al. (2018) (Microbial Ecology 75 (D1)) demonstrated that the inclusion of biomass at 2% OM inhibits methane production more effectively than an equivalent dose of manufactured bromoform in DMSO, indicating that the other components of Asparagopsis contribute to its anti-methanogenic activity.

As indicated above, bromoform is volatile, and so studies using volatile bromoform (e.g. un-stabilised) in closed systems have not demonstrated that manufactured bromoform could be delivered so as to be bioavailable in the rumen.

Importantly, more recent work has demonstrated that manufactured bromoform does not inhibit methane production (Stefenoni et al. (2019) J. Dairy Sci. Vol. 102, Suppl. 1, page 378). Without wishing to be bound by theory, the present inventors propose that bromoform must be made available in a form that is bioavailable in the rumen.

Exposure to high levels of concentrated bromoform are considered to be hazardous to animals, and synthetic bromoform and chemically related compounds (e.g. bromochloromethane) as manufactured/synthetic/purified chemicals are unsafe and disallowed for human and animal applications, including the inhibition of methanogenesis in ruminant animals.

Very recently, Muizelaar et al. Foods (2021) 10:584 published results presented data asserting that following feeding of Asparagopsis to cattle, bromoform was detected in the milk of some cows only on the first day of feeding in the low and medium treatment groups (9.1 and 11 μg/L, respectively), and detected in the milk of one single sample in the high treatment group only on day 9 (35 μg/L), and that two animals were sacrificed, and their rumen wall showed abnormalities. Upon histological examination, signs of inflammation became visible. The authors of that study conclude that within the confines of their experiment, CHBr₃ does not accumulate in animal tissue, but can be excreted in urine and milk. This paper expresses numerous concerns for human consumption following Asparagopsis use, and that further work is required to examine effects of ruminant animals consuming bromoform containing material. Notwithstanding that this paper teaches away from the use of bromoform containing materials, as part of the work described herein, the present inventors propose that the compositions of the present invention allow for uses of significantly lower amounts of bromoform.

The present inventors also consider that the work described in Muizelaar et al. does not link inflammation directly with Asparagopsis.

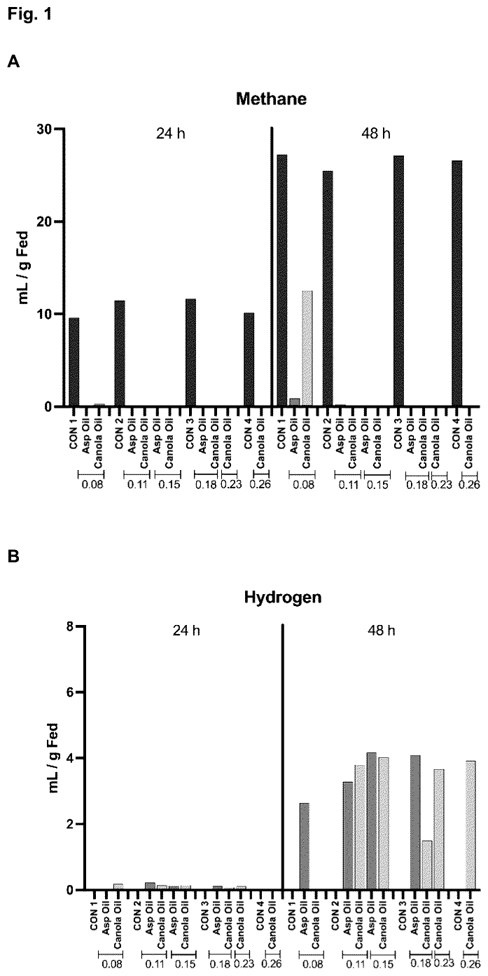

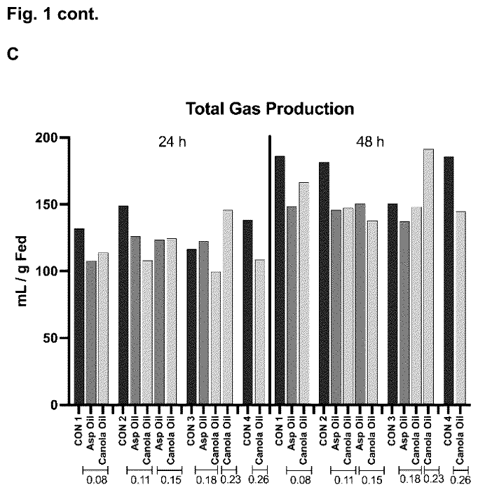

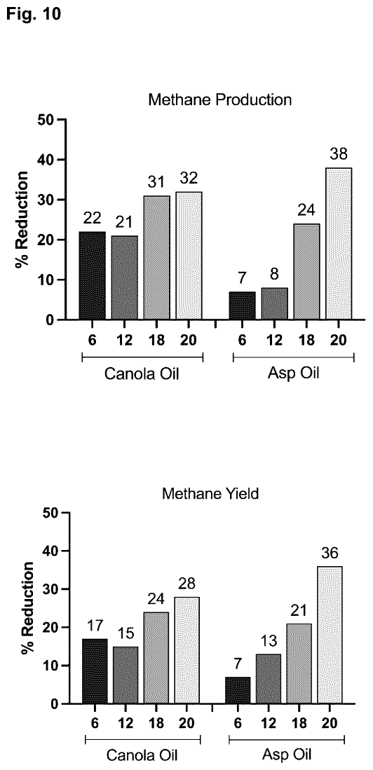

The present inventors have examined the effect of compositions comprising manufactured bromoform and a bromoform stabilising excipient. In particular, the present inventors have demonstrated in FIGS. 1, 2, 3, 6, and 9 that compositions comprising manufactured bromoform and a bromoform stabilising excipient can reduce methane produced in in vitro fermentation, and in FIG. 10 that the compositions described herein can inhibit methane production in vivo. In some embodiments, the compositions comprising manufactured bromoform and a bromoform stabilising excipient can eliminate methane production from in vitro and/or in vivo fermentation.

Importantly, the present inventors have demonstrated in Example 8 that the in vivo effects of the compositions described herein include improvement of growth performance. In particular, the in vivo effects observed include increased average daily weight gain, and increased feed utilisation efficiency.

In direct contrast to the data presented herein, previous work demonstrated that bromoform inclusion at doses of 0.25, 1 and 1.5% of feed dry matter had no effect on methane production (Stefenoni et al. (2019) J. Dairy Sci. Vol. 102, Suppl. 1, page 378, Dose-response effect of the macroalga Asparagopsis taxiformis on enteric methane emission in lactating dairy cows), whereas in the same experiments, Asparagopsis (which comprises a number of anti-methanogenic compounds, including bromoform), when included in in vitro fermentation, decreased methane (CH₄) yield linearly across doses of 0.25, 0.5, 0.75, 1.0 and 1.5% of feed dry matter. This indicates that bromoform alone has no effect on methane production, whereas Asparagopsis, included at corresponding doses, inhibits methane production.

Accordingly, in one aspect the present invention provides a method for reducing total gas production and/or methane production in a ruminant animal comprising the step of providing said ruminant animal with an effective amount of a composition comprising manufactured bromoform and a bromoform stabilising excipient.

Machado et al. (2016) J Appl Phycol 28:3117-3126 demonstrated that Asparagopsis taxiformis contains a number of anti-methanogenic compounds, including bromoform, dibromochloromethane, and bromochloromethane. This work demonstrated that manufactured bromoform formulated in DMSO significantly inhibits methane production at doses of 5 μM, 10 μM and 25 μM, but at 1 μM, manufactured bromoform does not significantly inhibit methane production in in vitro fermentation. Machado et al. (2016) indicates that bromoform at 1.3 μM is the amount of bromoform equivalent to the inclusion of Asparagopsis at 2% OM (24.7 mg) in vitro. Because 1 μM manufactured bromoform did not significantly inhibit methane production, Machado et al. indicates that for bromoform to significantly inhibit methane production, more than 1 μM bromoform is required. The compositions described herein allow the provision of compositions that are effective in reducing methane production at doses of bromoform significantly lower than those previously demonstrated to provide no significant effect in Machado et al. (2016) J Appl Phycol 28:3117-3126.

The present invention demonstrates that total gas production and/or methane production is reduced using 0.01 mg manufactured bromoform and a bromoform stabilising excipient per gram of feed substrate, and that that total gas production and/or methane production can be eliminated at higher doses of compositions comprising manufactured bromoform and a bromoform stabilising excipient (e.g. 0.04 mg or more of manufactured bromoform and a bromoform stabilising excipient). This data is in contrast to that of Machado et al which demonstrates that significantly more bromoform is required (e.g. more than 1 μM manufactured bromoform is required).

There are a number of problems with the use of Asparagopsis biomass and Asparagopsis derived products to inhibit methane production. One problem is that the amounts of secondary metabolites known to inhibit methane production (bromoform, dibromochloromethane, and bromochloromethane) vary between batches of Asparagopsis and so forming compositions from Asparagopsis that have the desired amounts of bromoform, dibromochloromethane, and bromochloromethane requires significant effort. In contrast to Asparagopsis biomass and Asparagopsis derived products, the compositions described herein allow the provision of compositions that comprise manufactured bromoform in controlled amounts.

In one embodiment the present invention provides a stabilised bromoform composition comprising manufactured bromoform and a bromoform stabilising excipient, wherein the bromoform stabilising excipient is selected from the group consisting of an edible oil, and edible carbohydrate and water.

In one embodiment, the stabilised bromoform compositions described herein do not comprise dibromochloromethane, bromochloroacetic acid, and/or dibromoacetic acid

Machado et al. (2016) indicates that bromoform at 1.3 μM is the amount of bromoform equivalent to the inclusion of Asparagopsis at 2% OM (24.7 mg) in vitro. Since more than 1 μM bromoform is required to significantly inhibit methane production-based on the bromoform content and the data of Machado et al.—inclusion of more than Asparagopsis at 2% OM (24.7 mg) would be required to significantly inhibit methane production. However, it is known that inclusion of Asparagopsis at high % levels of OM of feed can result in animals avoiding consuming the feed (Muizelaar, et al. Foods (2021) 10:584). The compositions described herein allow the provision of compositions that avoid the use of Asparagopsis biomass or Asparagopsis derived products that are unpalatable to ruminant animals.

Furthermore, and without wishing to be bound by theory, the present inventors propose that other secondary metabolites of Asparagopsis (including dibromochloromethane, and bromochloromethane) may contribute to an anti-methanogenic effect—when provided in combination—of Asparagopsis biomass or

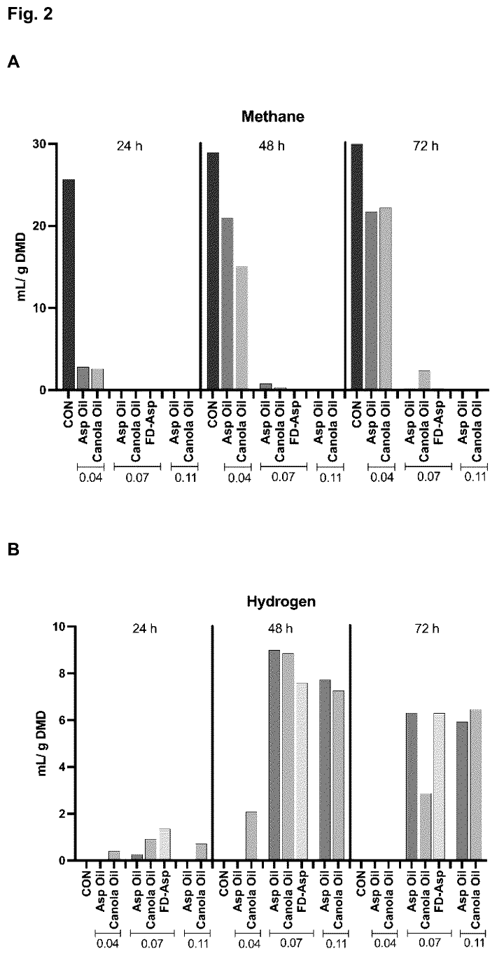

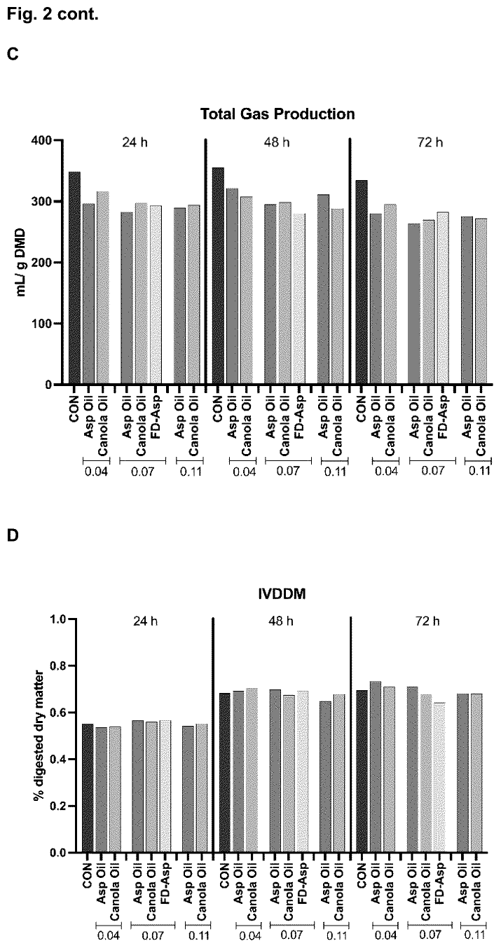

Asparagopsis derived products. For Example, Machado et al. (2016) J Appl Phycol 28:3117-3126 demonstrates in FIG. 2 that total gas production is reduced significantly by Asparagopsis biomass, but extracts at doses equivalent to the same amount of biomass do not reduce total gas production, and Asparagopsis biomass had the greatest inhibition of methane production compared to extracts equivalent to the same amount of biomass, indicating that other secondary metabolites contribute to the anti-methanogenic effect

Consistent with this preliminary data in FIG. 1 suggested that Asparagopsis biomass in oil inhibits methane production to a greater extent than compositions comprising manufactured bromoform at the same concentration (0.08 mg per g OM), and that Asparagopsis biomass in oil and manufactured bromoform in oil perform in the same manner inhibits methane production to the same extent at higher doses. Importantly however, the present inventors have now demonstrated that manufactured bromoform in stabilising excipients inhibit methane production to the same extent as Asparagopsis biomass in oil.

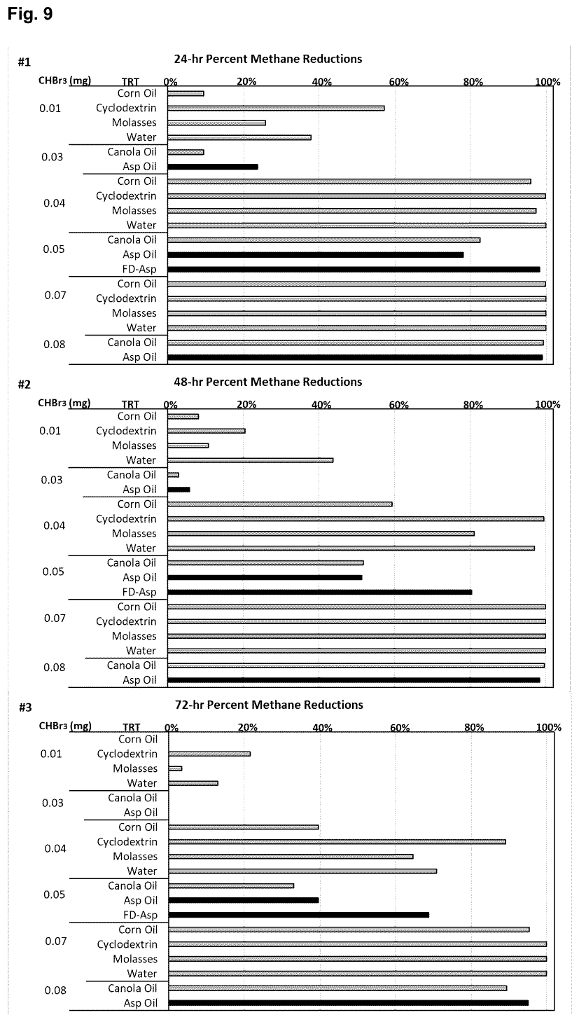

It remains unclear if the metabolites—when provided separately—can inhibit methanogenesis. For example, as discussed above, previous work demonstrated that manufactured bromoform did not inhibit methane production. The compositions described herein allow the provision of compositions that are effective in reducing methane production in the absence of dibromochloromethane, and bromochloromethane, and the other secondary metabolites of Asparagopsis. Without wishing to be bound by theory, the present inventors propose that the compositions described herein comprising manufactured bromoform and a bromoform stabilising excipient provide bioavailable bromoform that is able to inhibit methanogenesis at low levels. As summarised in FIG. 9, the present invention provides for the use of numerous classes of bromoform stabilising excipients that when combined with bromoform, provide bioavailable bromoform that is able to inhibit methanogenesis at low levels.

The present inventors have demonstrated in Example 6 that a composition comprising bromoform and a bromoform stabilising excipient, wherein the bromoform is Asparagopsis-derived bromoform, is able to inhibit methane production in animals. Example 8 demonstrates that the in vivo effects of the compositions described herein include improvement of growth performance measures. In particular, the in vivo effects observed include increased average daily weight gain, and increased feed utilisation efficiency. In another embodiment provided herein, the present invention provides a composition comprising bromoform and a bromoform stabilising excipient, wherein the bromoform is Asparagopsis-derived bromoform.

In one embodiment, the present invention provides a composition comprising bromoform and a bromoform stabilising excipient, wherein the bromoform is Asparagopsis-derived bromoform, wherein the composition further comprises manufactured bromoform.

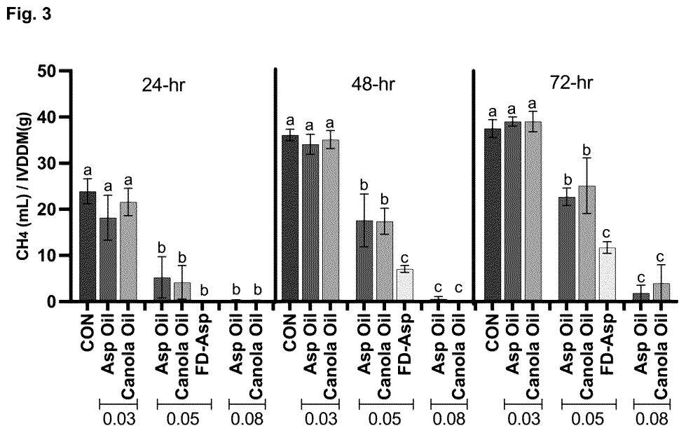

FIG. 3 sets out initial work showing that an Asparagopsis-derived composition comprising Asparagopsis biomass extracted into a bromoform stabilising excipient (Asp-Oil) inhibits methanogenesis to the same extent as a manufactured bromoform in Canola Oil despite the canola oil composition not containing the other anti-methanogenic compounds present in Asp-Oil. The ability to deliver bioavailable manufactured bromoform at controlled amounts, in the absence of the other contaminants of Asparagopsis, allows new uses of the compositions described herein.

A further problem is increasing the inclusion level of Asparagopsis biomass or Asparagopsis derived products also requires attention to trace elements and minerals that may accumulate in seaweeds. Asparagopsis, like many other seaweeds, may contain iodine at concentrations that may exceed maximum tolerable levels in feeding systems. The compositions comprising manufactured bromoform and a bromoform stabilising excipient described herein allow trace elements and minerals of Asparagopsis biomass or Asparagopsis derived products, such as iodine, to be omitted.

Furthermore, Asparagopsis, like many other seaweeds, may contain bromine at concentrations that may exceed maximum tolerable levels in feeding systems. The compositions comprising manufactured bromoform and a bromoform stabilising excipient described herein allow trace elements and minerals of Asparagopsis biomass or Asparagopsis derived products, such as bromine, to be omitted.

As used herein, the term “reducing” includes the reduction of amount of substance in comparison with a reference. For example, the reduction in the amount of total gas and/or methane produced by a ruminant animal or animals administered an effective amount of a composition comprising manufactured bromoform and a bromoform stabilising excipient according to the present invention, relative to an animal or animals not administered a composition comprising an effective amount of a composition comprising manufactured bromoform and a bromoform stabilising excipient as described herein. The reduction can be measured in vitro with an artificial rumen system that simulates anaerobic fermentation, or in vivo with animals confined in respiration chambers. It is within the knowledge and skill of those trained in the art to assess enteric methanogenesis by a ruminant animal.

As used herein the term “anaerobic fermentation” is intended to include anaerobic fermentation in vitro, for example in experimental systems, and in vivo, for example, in a ruminant animal.

As used herein, the term ‘reducing total gas production’ refers to the reduction of the total amount of gas produced, for example the amount of total gas produced in the gastro-intestinal tract. The term includes the collective volume of all gasses generated as a result of anaerobic fermentation, for example, in the systems described herein. Fermentation in the rumen and the gut of a ruminant gives rise to production of gas including methane. The present invention aims to reduce this process, such as to reduce the total amount of gas produced in the gastro-intestinal tract. It is within the knowledge and skill of those trained in the art to assess total gas production by a ruminant animal.

As used herein, the term ‘reducing methane production’ refers to the reduction of methane produced in the gastro-intestinal tract. The term includes the specific volume of methane generated as a result of anaerobic fermentation, for example, in the systems described herein. Fermentation in the rumen and the gut of a ruminant gives rise to production of methane. The present invention aims to reduce this process, such as to reduce the total amount of methane produced in the gastro-intestinal tract. It is within the knowledge and skill of those trained in the art to assess methane production by a ruminant animal.

In preferred embodiments of the invention, the amount of total gas produced is reduced by at least 95%, 90%, 80%, 70%, 60%, 50%, 40%, 30%, 20% or 10% compared to a reference. In one embodiment the reference is the amount of total gas produced when animals are not administered an effective amount of a composition comprising manufactured bromoform and a bromoform stabilising excipient. In another embodiment, the reference is the amount of total gas produced when animals are administered a control feed. In another embodiment, the reference is the amount of total gas produced when a control feed is subjected to in vitro anaerobic fermentation.

In preferred embodiments of the invention, the amount of methane produced is reduced by at least 95%, 90%, 80%, 70%, 60%, 50%, 40%, 30%, 20% or 10% compared to a reference. In one embodiment the reference is the amount of methane produced when animals are not administered an effective amount of a composition comprising manufactured bromoform and a bromoform stabilising excipient. In another embodiment, the reference is the amount of methane produced when animals are administered a control feed. In another embodiment, the reference is the amount of methane produced when a control feed is subjected to in vitro anaerobic fermentation.

By “effective amount”, is meant a quantity of a composition comprising manufactured bromoform and a bromoform stabilising excipient as described herein sufficient to allow improvement, e.g. reduction in the amount of methane production in comparison with a reference or control, reduction in the amount of total gas produced in comparison with a reference or control, maintenance of effective levels of desirable volatile fatty acids in comparison with a reference or control, reduction in the acetate to propionate ratio in comparison with a reference or control, maintenance of liveweight, dry matter intake and/or organic matter intake in comparison with a reference or control, or improvement in growth performance relative in comparison with a reference or control. Within the meaning of the present invention, the methane reductive effect can be measured in the rumen with an artificial rumen system, such as that described in T. Hano., J. Gen. Appl. Microbiol., 39, 35-45, 1993 or by in vivo oral administration to ruminants.

An effective amount of a composition comprising manufactured bromoform and a bromoform stabilising excipient may be determined by the methods described herein, including the in vitro and in vivo studies and in vivo dose-response studies described in Kinley et al. (2020) Journal of Cleaner Production 259:120836 and WO2015109362, incorporated herein by reference. For example, the present inventors have demonstrated that ruminal fermentation in vitro can be used to examine the effect of amounts of a composition comprising manufactured bromoform and a bromoform stabilising excipient on total gas, hydrogen and methane production, and can be used to examine levels of volatile fatty acids, including acetate and propionate. Measures of growth performance can be measured using the in vivo studies described herein. Therefore, ruminal fermentation in vitro can be used to characterize doses of the composition comprising manufactured bromoform and a bromoform stabilising excipient that may be an effective amount sufficient to allow improvement, e.g. reduction in the amount of methane production in comparison with a reference or control, reduction in the amount of total gas produced in comparison with a reference or control, maintenance of effective levels of desirable volatile fatty acids in comparison with a reference or control, or reduction in the acetate to propionate ratio in comparison with a reference or control.

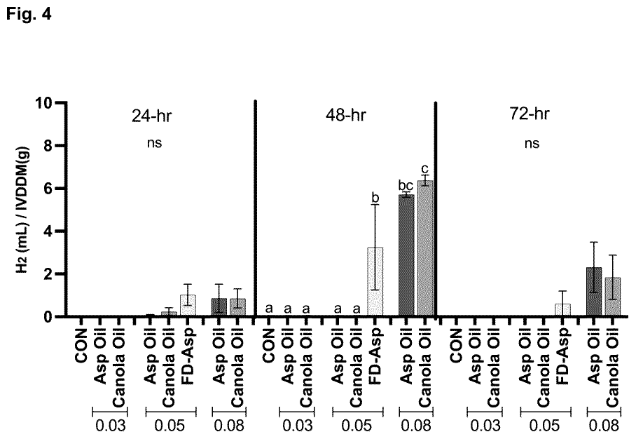

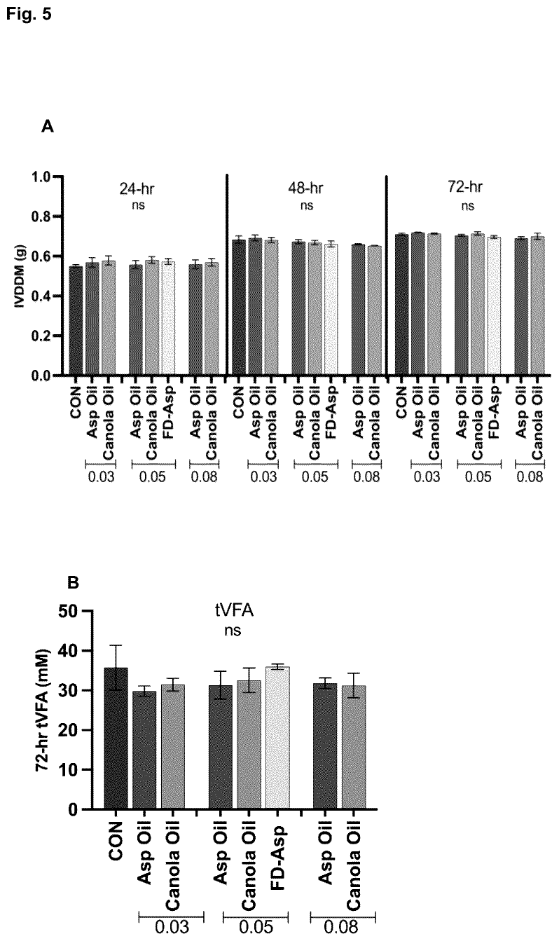

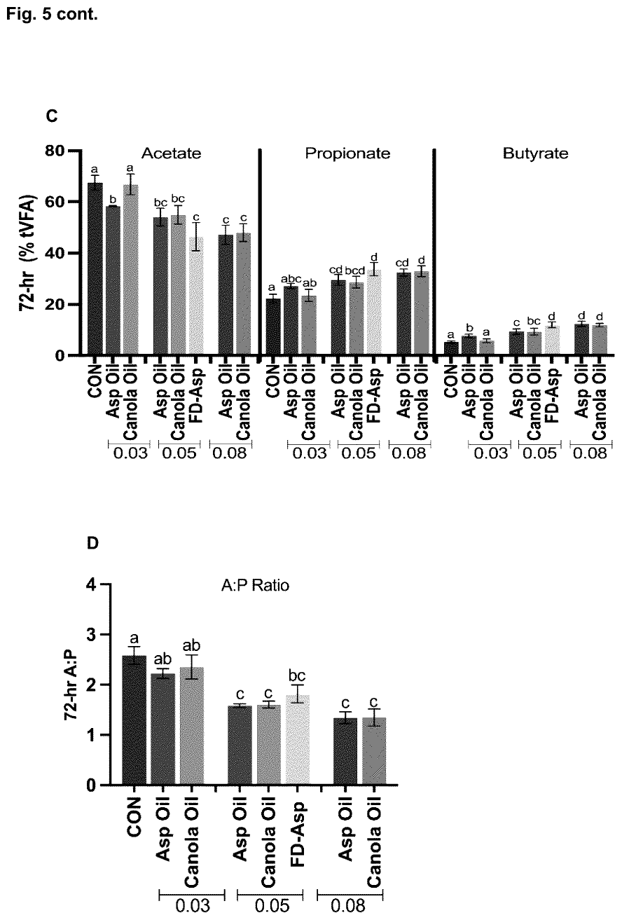

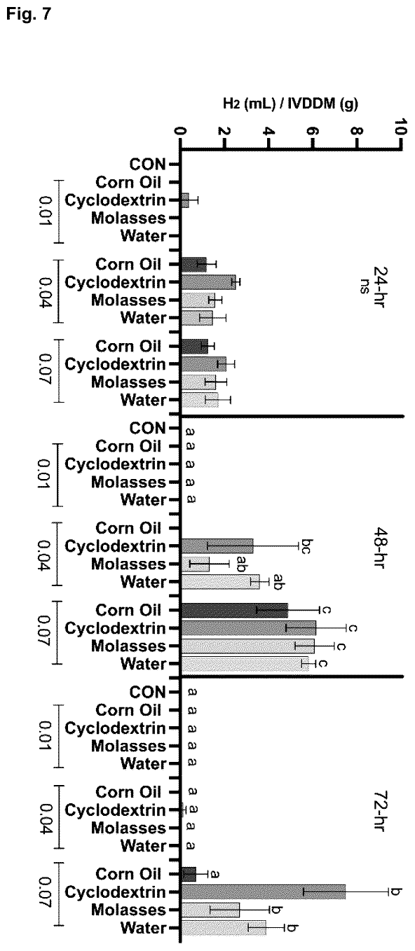

For example, FIGS. 4 and 7 shows that at high doses, compositions comprising manufactured bromoform and bromoform stabilising excipients can increase hydrogen levels, consistent with the inhibition of methane production. FIG. 5 demonstrates no negative impacts to fermentation were observed on IVDDM and tVFA production.

As used herein the term “providing” includes the provision of a composition comprising manufactured bromoform and a bromoform stabilising excipient as a feed additive in feed provided to an animal or to an animal system, (e.g. animals in a feedlot, or animals in a farming system, etc). Providing also includes the administration of a composition as described herein.

In one embodiment, the farming system is a pastoral, feedlot, regularly supplemented system, or a combination thereof.

By “administer” and “administered”, is meant the action of introducing a composition as described herein into the animal's gastro-intestinal tract. More particularly, this administration is an administration by oral route. This administration can in particular be carried out by supplementing the feed intended for the animal with the composition comprising manufactured bromoform and a bromoform stabilising excipient, the thus supplemented feed then being ingested by the animal. This administration can also include providing the composition comprising manufactured bromoform and a bromoform stabilising excipient in a form that an animal consumes (e.g. a lick block, supplement, etc). The administration can also be carried out using a stomach tube or any other means making it possible to directly introduce said composition (e.g. a dosage form (e.g. a bolus) of composition) into the animal's gastro-intestinal tract.

Accordingly, the present invention provides a method as described herein wherein the step of providing said ruminant animal an effective amount of a composition comprises administering said ruminant animal with the effective amount of a composition comprising manufactured bromoform in a bromoform stabilising excipient.

The composition comprising manufactured bromoform and a bromoform stabilising excipient may be provided to the ruminant in one of many ways. A composition comprising manufactured bromoform and a bromoform stabilising excipient can be provided in a solid form as a veterinary pharmaceutical, may be distributed in an excipient, and directly fed to the animal, may be physically mixed with feed material in any suitable form (e.g. dry form, in solution, or in suspension etc.) or the composition comprising manufactured bromoform and a bromoform stabilising excipient may be formed into a solution and thereafter sprayed onto feed material. For example, a dry mixture can be used that is prepared by adsorption or deposition of a solution onto/into a dry excipient.

The method of administration of the composition comprising manufactured bromoform and a bromoform stabilising excipient to the animal is considered to be within the skill of the artisan.

When used in combination with a feed material, the feed material is preferably grain/hay/silage/grass-based. Included amongst such feed materials are improved and/or grass or legume-based forages either grazed directly or prepared as a conserved forage hay, any feed ingredients and food or feed industry by-products as well as bio-fuel industry by-products and corn meal and mixtures thereof, or feed lot and dairy rations, such as those high in grain content.

The composition comprising manufactured bromoform and a bromoform stabilising excipient may be provided for consumption in an animal system (e.g. a feedlot, a native grass land/pasture farming system), in an animal feed supplement as an inclusion in any suitable form, including, a mineral loose lick, wet lick, pellets, water suspension, or lick block. As is known to those skilled in the art such loose licks, wet licks, pellets, water suspensions, or lick blocks are particularly convenient for feeding mineral supplements (as well as proteins and carbohydrates) to ruminants grazing pastures. Such loose licks, wet licks, pellets, water suspensions, or lick blocks etc. may comprise, in addition to the composition comprising manufactured bromoform and a bromoform stabilising excipient of the invention, various types of binders, e.g. cements, gypsum, lime, calcium phosphate, carbonate, and/or gelatin; and optionally further additives such as vitamins, trace elements, mineral salts, sensory additives, etc.

The time of administration is not crucial so long as the reductive effect on methane production and/or growth performance is shown. As long as the feed is retained in the rumen, administration is possible at any time. However, since the composition comprising manufactured bromoform and a bromoform stabilising excipient is preferably present in the rumen at about the time methane is produced, the composition comprising manufactured bromoform and a bromoform stabilising excipient is preferably administered with or immediately before feed.

In a particular embodiment of the invention, said effective amount of the composition comprising manufactured bromoform and a bromoform stabilising excipient is administered to a ruminant animal by supplementing a feed intended for said animal with the composition comprising manufactured bromoform and a bromoform stabilising excipient. By “supplementing”, within the meaning of the invention, is meant the action of incorporating the effective amount of the composition comprising manufactured bromoform and a bromoform stabilising excipient according to the invention directly into the feed intended for the animal. Thus, the animal, when feeding, ingests the composition comprising manufactured bromoform and a bromoform stabilising excipient according to the invention which can then act to maintain the digestibility of the fibres and/or cereals contained in the animal's feed. Alternatively, supplements, such as loose lick, wet lick, pellets, water suspension, or lick blocks and other feed supplements, can be provided in an animal system without incorporating directly into animal feed.

Thus, another subject of the invention relates to a feed supplement for a ruminant animal comprising a composition comprising manufactured bromoform and a bromoform stabilising excipient.

In another aspect the present invention also provides a feed supplement for reducing total gas production and/or methane production in a ruminant animal, said supplement comprising an effective amount of a composition comprising manufactured bromoform and a bromoform stabilising excipient.

In one embodiment, the effective amount of a composition comprising manufactured bromoform and a bromoform stabilising excipient is administered to said ruminant animal by supplementing food intended for said animal with said composition comprising manufactured bromoform and a bromoform stabilising excipient.

As discussed above, the present inventors have demonstrated in Example 6 that a composition comprising bromoform and a bromoform stabilising excipient, wherein the bromoform is Asparagopsis-derived bromoform, is able to inhibit methane production in vivo. Example 8 demonstrates that the in vivo effects of the compositions described herein include improvement of growth performance measures. In particular, the in vivo effects observed include increased average daily weight gain, and increased feed utilisation efficiency.

Accordingly, in one embodiment the present invention provides methods for improving the growth performance of a ruminant animal comprising the step of providing said ruminant animal with an effective amount of a composition comprising manufactured bromoform and a bromoform stabilising excipient.

In a preferred embodiment, the growth performance is increased average daily weight gain and/or increased feed utilisation efficiency.

In one embodiment the present invention provides a method of increasing average daily weight gain of a ruminant animal comprising the step of providing said ruminant animal with an effective amount of a composition comprising manufactured bromoform and a bromoform stabilising excipient.

In one embodiment the present invention provides a method of increasing feed utilisation efficiency of a ruminant animal comprising the step of providing said ruminant animal with an effective amount of a composition comprising manufactured bromoform and a bromoform stabilising excipient.

In one embodiment, the present invention provides a method as described herein wherein the effective amount of a composition comprising manufactured bromoform and a bromoform stabilising excipient is provided in the farming system, thereby reducing total gas production and/or methane production and/or improving the growth performance of the livestock animal in the farming system.

In one embodiment, the present invention provides a method as described herein wherein the effective amount of a composition comprising manufactured bromoform and a bromoform stabilising excipient is provided to a stocker operation to enable consumption of the composition comprising manufactured bromoform and a bromoform stabilising excipient by a livestock animal in the stocker operation, thereby reducing total gas production and/or methane production and/or improving the growth performance of the livestock animal in stocker operation.

In one embodiment, the present invention provides a method as described herein wherein the effective amount of a composition comprising manufactured bromoform and a bromoform stabilising excipient is provided to a feedlot system to enable consumption of the composition comprising manufactured bromoform and a bromoform stabilising excipient by a livestock animal in the feedlot system, thereby reducing total gas production and/or methane production and/or improving the growth performance of the livestock animal in the feedlot system.

In one embodiment, the present invention provides a method as described herein wherein the effective amount of a composition comprising manufactured bromoform and a bromoform stabilising excipient is provided in the pasture system to enable consumption of the composition comprising manufactured bromoform and a bromoform stabilising excipient by a livestock animal in the pasture system thereby reducing total gas production and/or methane production and/or improving the growth performance of the livestock animal in the pasture system.

A ruminant is a mammal of the order Artiodactyla that digests plant-based food by initially softening and partially fermenting it within the animal's first stomach chambers, then regurgitating the semi-digested mass, now known as cud, and chewing it again.

The process of rechewing the cud to further break down plant matter and stimulate digestion is called “ruminating”. Ruminants have a digestive tract with four chambers, namely the rumen, reticulum, omasum and abomasum. In the first two chambers, the rumen and the reticulum, the food is mixed with saliva and separates into layers of solid and liquid material. Solids clump together to form the cud, or bolus. The cud is then regurgitated, chewed slowly to completely mix it with saliva, which further breaks down fibers. Fiber, especially cellulose, is broken down into glucose in these chambers by symbiotic anaerobic bacteria, protozoa and fungi in the unique process of rumen microbial digestive fermentation. The broken-down fiber, which is now in the liquid part of the contents, then carried out of the rumen into the next stomach chamber, the omasum where further fermentation occurs and water and solubilised nutrients are absorbed. The next phase is the abomasum where undigested and rumen bypassed feed is digested much like it would be in the monogastric stomach. Digested gut contents are finally sent to the small intestine, where the absorption of the nutrients occurs. Almost all the glucose produced by the fermentation of cellulose is used by the symbiotic microbial consortium. Ruminants get their energy from the volatile short chain fatty acids (VFAs) produced by the bacteria, namely acetate, propionate, butyrate, valerate, and isovalerate. Most of the protein utilised by the ruminant animal is microbial protein. A by-product of bacterial fermentation of feed is CO₂ and H₂ which are used in a reductive process by anaerobic archaea which in turn have the waste by-product of CH₄. Production of CH₄ is an energy inefficiency of the digestive fermentation of ruminant animals.

Importantly, the inventors have shown that the compositions described herein possess the property of reducing total gas production and/or methane production in ruminant animals without compromising rumen fermentation as measured by dry matter digestibility.

Examples of ruminants are listed below. However, preferably compositions described herein are used as an additive for foodstuffs for domesticated livestock such as cattle, goats, sheep and llamas. The present invention is particularly useful in cattle and sheep. Therefore, in one embodiment, said ruminant animal is selected from the members of the Ruminantia and Tylopoda suborders. In another embodiment, said ruminant animal is cattle or sheep. In a further embodiment, said ruminant animal is a cattle.

Bromoform (CHBr₃) is also referred to by a number of synonyms, including tribromomethane-see PubChem CID: 5558.

As used herein the term “manufactured bromoform” refers to bromoform not derived from Asparagopsis biomass, and the term includes artificially synthesized bromoform. The manufactured bromoform can be artificially synthesised by chemical means by known methods, including a haloform reaction using acetone and sodium hypobromite, electrolysis of potassium bromide in ethanol, or by treating chloroform with aluminium bromide, or reacting chloroform with sodium hydroxide to yield bromoform and sodium chloride.

Manufactured bromoform can be also be artificially synthesised by biological means, for example using enzymes, or genetically modified organisms, including in a manufacturing system, and the bromoform produced collected from headspace gas or trapped using a chemical or mechanical means. For example, WO2020243792 (incorporated herein by reference), describes recombinant yeast capable of producing bromoform.

In a preferred embodiment, the manufactured bromoform is free bromoform provided in a purified form. For example, in one embodiment the manufactured bromoform is 90% pure, and may comprise low levels (e.g. 1-10%) of a stabilizer, such as ethanol or amylene and the like. In another embodiment, the manufactured bromoform is at least 90% pure, at least 91% pure at least 92% pure, at least 93% pure, at least 94% pure, at least 95% pure, at least 96% pure, at least 97% pure, at least 98% pure or at least 99% pure.

In a preferred embodiment, the compositions described herein do not comprise iodine.

In a preferred embodiment, the compositions described herein do not comprise bromine.

In one embodiment, the present invention provides a stabilised bromoform composition comprising manufactured bromoform and a bromoform stabilising excipient, wherein the bromoform stabilising excipient is selected from the group consisting of an edible solid, edible semi solid or edible liquid.

In one embodiment, the present invention provides a stabilised bromoform composition as described herein, wherein the bromoform stabilising excipient is selected from the group consisting of an edible oil, an edible carbohydrate and water.

In one embodiment, the present invention provides a stabilised bromoform composition as described herein, wherein the composition does not comprise one or more compounds selected from the group consisting of iodine, bromine, dibromochloromethane, bromochloroacetic acid, and dibromoacetic acid.

In one embodiment, the present invention provides a method of making a stabilised bromoform composition comprising contacting manufactured bromoform and a bromoform stabilising excipient, wherein the bromoform stabilising excipient is selected from the group consisting of an edible solid, edible semi solid or edible liquid.

In one embodiment, the present invention provides a method of making a stabilised bromoform composition as described herein, wherein the bromoform stabilising excipient is selected from the group consisting of an edible oil, an edible carbohydrate and water.

In one embodiment, the present invention provides a method of making a stabilised bromoform composition, wherein the composition does not comprise one or more compounds selected from the group consisting of iodine, bromine, dibromochloromethane, bromochloroacetic acid, and dibromoacetic acid.

The present inventors have demonstrated herein that stabilising excipients can be used to stabilise volatile bromoform to be bioavailable to ruminal fermentation to inhibit methanogenesis. Importantly, the present inventors have demonstrated that carbohydrates (including cyclodextrin and molasses), water, and edible non-polar substances (such as oils) can be used to stabilise bromoform and allow for the formation of compositions that make bromoform bioavailable to ruminal fermentation.

Stabilising excipients are discussed below, and including edible solids, liquids and semi-solids that are able to stabilise bromoform.

Importantly, the ability to use different stabilising excipients allows for the formation of compositions that are solid, semi solid or liquid, and which comprise bromoform that is bioavailable to ruminal fermentation.

Furthermore, the ability to use different stabilising excipients allows for the formation of compositions with defined stability.

For example, FIG. 10 shows the stability of the compositions described herein over time in different storage conditions.

FIG. 10 also shows that the sustained release characteristics of the compositions can be determined, including at different conditions. Accordingly, these sustained released characteristics can be used to provide methods of improving growth performance or reducing methane production that account for different times to feed uptake and/or different temperatures (e.g. climates).

It has been surprisingly found by the present inventors that stabilising excipients combined with manufactured bromoform delays release of volatile manufactured bromoform, providing prolonged or sustained release of the manufactured bromoform.

The term “volatile” used herein refers to the tendency of the agent to evaporate and generally refers to fluid substances.

The term “such that sustained release of the agent is provided” means that the manufactured bromoform and the stabilising excipient are capable of dissociation whereupon the manufactured bromoform is released at a reduced rate compared to the manufactured bromoform administered on its own.

Accordingly in one aspect the invention provides an anti-methanogenic composition for use in animals comprising manufactured bromoform together with a stabilising excipient such that sustained release of the manufactured bromoform is provided.

In another aspect the invention provides an animal feed comprising a composition of the invention described above together with a nutrient source.

In another aspect the present invention provides a medicament for administering manufactured bromoform to an animal over an extended period comprising a composition comprising manufactured bromoform together with a stabilising excipient such that sustained release of the manufactured bromoform is provided, in a manner such that said composition is retained by said animal over said period.

The term “over an extended period” refers to a period of time which is longer than the time taken for the manufactured bromoform to evaporate when it is not present in the composition.

The term “in a manner such that said composition is retained by said animal over said period” means that the composition is applied in a suitable manner to allow sustained release. In a ruminant for example administration may be provided in the form of a controlled release device or may be provided in the feed.

Where the animal is a ruminant the method leads to increased weight gains (e.g. increased average daily weight gains) as demonstrated herein. The method is also of benefit for anerobic fermentation of ruminant and non-ruminant manure in that it reduces greenhouse gas emissions.

In a particularly preferred embodiment, the invention relates to a method of reducing methane production in an animal over an extended period comprising administering a methane reducing effective amount of an antimethanogenic composition said composition comprising manufactured bromoform together with a stabilising excipient such that sustained release of said agent is provided.

As used herein the term “stabilising excipient” refers to an excipient which prevents loss of bromoform from the composition, and which allows the manufactured bromoform to be bioavailable in a ruminant animal. Stabilising excipients include those described herein, including edible solids, liquids and semi-solids that are able to stabilise bromoform.

Suitable stabilising excipients include alcohol (e.g. ethanol), benzene, chloroform, ether, petroleum ether, DCM, diethyl ether, hexane, pyridine, toluene, xylene, acetone and oils. Preferably, the bromoform stabilising excipient is safe for consumption by animals at final concentration in the dose of the composition to be provided to animals.

In a preferred embodiment, the bromoform stabilising excipient allows the manufactured bromoform to be bioavailable in a ruminant animal when the composition comprises manufactured bromoform at low doses.

In a preferred embodiment, the bromoform stabilising excipient allows the manufactured bromoform to be bioavailable in a ruminant animal when the composition comprises manufactured bromoform at a dose of less than 5 μM bromoform.

In a preferred embodiment, the bromoform stabilising excipient allows the manufactured bromoform to be bioavailable in a ruminant animal when the composition comprises manufactured bromoform at a dose of less than 1 μM bromoform.

In a preferred embodiment, the bromoform stabilising excipient is not DMSO.

In one aspect the present invention provides a method of making a stabilised bromoform composition comprising contacting manufactured bromoform and a bromoform stabilising excipient, wherein the bromoform stabilising excipient is selected from the group consisting of an edible oil, and edible carbohydrate and water.

In one aspect the present invention provides a process for preparing a composition comprising manufactured bromoform and a bromoform stabilising excipient composition, said process comprising the steps of:
(a) providing manufactured bromoform;
(b) providing a bromoform stabilising excipient; and
(c) contacting the manufactured bromoform with the bromoform stabilising excipient under conditions to form the composition.

In one embodiment, the bromoform stabilising excipient is selected from the group consisting of an edible wax, grease, oil, cyclodextrins, molasses and a saturated fat.

In one embodiment, the bromoform stabilising excipient is an edible protein.

The present inventors have demonstrated herein that edible carbohydrates, including cyclodextrins and molasses, can be used as stabilising excipients.

Accordingly, in one embodiment, the stabilising excipient is a carbohydrate.

Carbohydrates include molasses, cyclodextrins, lactose, dextrose, sucrose, glucose, fructose, galactose, xylose, arabinose, beta-glucans, galactans, pectins, and the like.

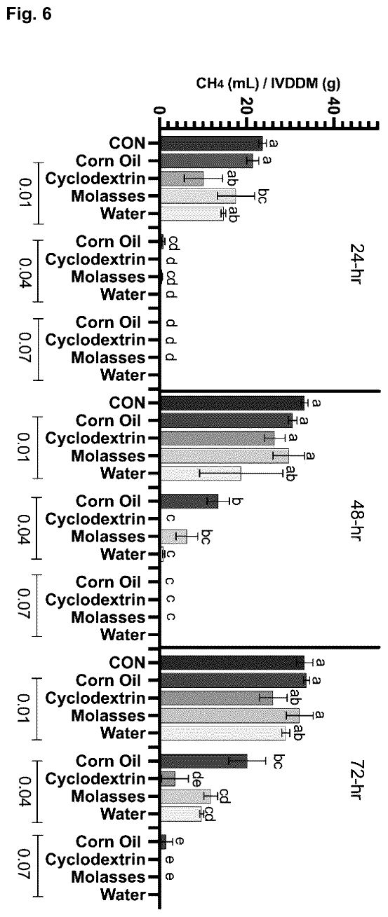

FIGS. 6 and 9 demonstrate that a composition comprising manufactured bromoform and cyclodextrin as the bromoform stabilising excipient can be formed, and can inhibit methane production from ruminal fermentation.

In another embodiment, the stabilising excipient is a cyclodextrin.

Cyclodextrins (sometimes called cycloamyloses) are cyclic oligosaccharides that contain glucose units, i.e., (α-1,4)-linked a-D-glucopyranose units, bound together in a ring. Cyclodextrins are typically produced from starch by means of enzymatic conversion. Typical cyclodextrins contain a number of glucose monomers ranging from six to eight units in a ring, creating a cone or hollow stopper shape; for example, a (alpha)-cyclodextrin comprises a 6-membered sugar ring molecule, β (beta)-cyclodextrin comprises a 7-membered sugar ring molecule; and γ (gamma)-cyclodextrin comprises an 8-membered sugar ring molecule. The cyclodextrins suitable for the compositions of the present invention can, if desired, be modified by the addition of substituents. As used herein, “cyclodextrins” include both modified and unmodified cyclodextrins. Substituents generally replace either the entire hydroxyl group or the hydrogen atom on one or more of the hydroxyl groups of the cyclodextrin ring.

The cyclodextrin may be selected from α-cyclodextrin, β-cyclodextrin or γ-cyclodextrin or derivatives thereof which may be naturally and/or synthetically produced.

In one embodiment, the present invention provides a composition as described herein, wherein the composition is prepared by a method comprising the steps of:
(a) contacting manufactured bromoform and a cyclodextrin aqueous solution; and
(b) preparing a solid from step a).

In another embodiment, the present invention provides a composition described herein is prepared by a method comprising the steps of:
(a) contacting manufactured bromoform and a cyclodextrin aqueous solution;
(b) preparing a solid from step a); and
(c) preparing a powder from the solid from step b).

In another embodiment, the stabilising excipient is a molasses.

FIGS. 6, 9 and 10 demonstrate that a composition comprising manufactured bromoform and molasses as the bromoform stabilising excipient can be formed, and can inhibit methane production from ruminal fermentation in vitro and in vivo.

As used herein, “molasses” includes a syrup produced as a by-product of processing sugar cane or other vegetable products. A few examples include sugarcane waste (by-product of sugar production from sugarcane); high test (cane) molasses (primary product squeezed from sugarcane); sugarcane molasses (By-product of the process of refining unrefined brown sugar into white sugar); Sugar radish molasses (byproduct when sugar is produced from sugar beet); Citrus molasses (fruit juice squeezed in the production of dried citrus pulp); amongst others. In one embodiment, the bromoform stabilising excipient is an edible non-polar substance.

FIGS. 1, 2, 6, 9 and 10 demonstrate that a composition comprising manufactured bromoform and an edible oil as the bromoform stabilising excipient can be formed, and can inhibit methane production from ruminal fermentation.

FIG. 5 demonstrates that a composition comprising manufactured bromoform and an edible oil as the bromoform stabilising excipient can be formed, without negatively impacting ruminal fermentation, for example as measured by IVDDM and tVFA production). For example, in one embodiment the edible non-polar substance is selected from the group consisting of an oil or molasses.

In another embodiment, the edible non-polar substance is an edible oil.

As used herein the term “edible oil” includes a single type of oil, or compositions comprising a single type of oil, or a mixture of two or more oils, or a composition comprising a mixture of two or more oils. The oil includes an oil suitable for provision to, administration to, an animal.

In another embodiment, the edible non-polar substance is an edible fat that is solid at ambient temperature.

FIGS. 6 and 9 demonstrate that a composition comprising manufactured bromoform and water as the bromoform stabilising excipient can be formed, and can inhibit methane production from ruminal fermentation.

This result is surprising, since bromoform is considered to be only slightly soluble in water, and was considered to be soluble at less than 0.1 g bromofrm per 100 g water at 20 degrees (Mackison, F. W., R. S. Stricoff, and L. J. Partridge, Jr. (eds.). NIOSH/OSHA-Occupational Health Guidelines for Chemical Hazards. DHHS (NIOSH) Publication No. 81-123 (3 VOLS). Washington, DC: U.S. Government Printing Office, January 1981, p. 2).

In another embodiment, the bromoform stabilising excipient is water. The present inventors have prepared bromoform in water at a concentration of 3.0±0.10 mg bromoform per gram deionised water.

In one embodiment, the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil or any combination thereof.

As used herein, the term “oil” means any non-polar, hydrophobic substance which is typically a liquid at ambient temperature and pressure. Oils may be derived from animals, vegetables, or petrochemicals, and typically have a high carbon and hydrogen content. The oil is preferably an edible oil, and preferably digestible by a ruminant animal. Typically, an oil of vegetable origin is extracted from the seeds or fruits of plants, and is typically comprised primarily of triglycerides. The term “vegetable oil” is a generic term to indicate that the oil is of primarily or exclusively vegetable origin, and may comprise a mixture of one or more oils of vegetable origin or from differing origins.

As used herein the term “contacting” includes mixing the manufactured bromoform with the bromoform stabilising excipient to form a manufactured bromoform and bromoform stabilising excipient mixture, and retaining the manufactured bromoform, and retaining the manufactured bromoform and bromoform stabilising excipient composition for a period of time and at a temperature suitable to form the composition.

The term contacting includes retaining the manufactured bromoform and bromoform stabilising excipient at different suitable temperatures.

The temperature at which the manufactured bromoform is contacted with the bromoform stabilising excipient can be heated to is not limited, insofar that the manufactured bromoform does not evaporate/sublime appreciably from the bromoform stabilising excipient, and/or does not degrade appreciably at the temperature at which the heating takes place.

The time for which the manufactured bromoform is contacted with the bromoform stabilising excipient contacted is not limited, insofar that the manufactured bromoform does not evaporate/sublime appreciably from the bromoform stabilising excipient, and/or does not degrade appreciably during the time of contacting.

The present inventors have demonstrated herein that the compositions described herein maintain levels of volatile manufactured bromoform over extended periods of time.

For example, Example 7 demonstrates that compositions comprising manufactured bromoform and corn oil as the bromoform stabilising excipient maintain bromoform for four weeks in storage at −20° C., 4° C., 25° C. and 40° C.

Example 7 also demonstrates that compositions comprising manufactured bromoform and corn oil as the bromoform stabilising excipient maintain more than 46% of the starting bromoform concentration over four weeks in open air conditions at 25° C. and 40° C.

Example 7 demonstrates that compositions comprising manufactured bromoform and molasses as the bromoform stabilising excipient maintain bromoform for four weeks in storage at −20° C., 4° C., 25° C. and 40° C.

Example 7 demonstrates that compositions comprising manufactured bromoform and molasses as the bromoform stabilising excipient maintain bromoform for four weeks in storage at −20° C., and 4° C.

Example 7 also demonstrates that compositions comprising manufactured bromoform and molasses as the bromoform stabilising excipient maintain more than 38% of the starting bromoform concentration over four weeks in storage 25° C.

Example 7 also demonstrates that compositions comprising manufactured bromoform and molasses as the bromoform stabilising excipient maintain more than 24% of the starting bromoform concentration over four weeks in storage at 40° C.

Surprisingly, Example 7 also demonstrates that compositions comprising manufactured bromoform and molasses as the bromoform stabilising excipient maintain more than 58% of the starting bromoform concentration over four weeks in open air conditions at 25° C.

Also surprisingly, Example 7 also demonstrates that compositions comprising manufactured bromoform and molasses as the bromoform stabilising excipient maintain more than 62% of the starting bromoform concentration over four weeks in open air conditions at 40° C.

Example 7 also demonstrates that compositions comprising manufactured bromoform and cyclodextrin as the bromoform stabilising excipient maintain the starting bromoform concentration over four weeks in storage −20° C., 4° C., 25° C. and 40° C.

Example 7 also demonstrates that compositions comprising manufactured bromoform and cyclodextrin as the bromoform stabilising excipient maintain the starting bromoform concentration over four weeks in open air conditions at 25° C. and 40° C.

In one embodiment the present invention provides a stabilised bromoform composition comprising manufactured bromoform and a bromoform stabilising excipient.

In one aspect, the present invention provides a composition as described herein, wherein the level of manufactured bromoform in the composition is maintained following one week in storage at −20° C. In one aspect, the present invention provides a composition as described herein, wherein the level of manufactured bromoform in the composition is maintained following two weeks in storage at −20° C. In one aspect, the present invention provides a composition as described herein, wherein the level of manufactured bromoform in the composition is maintained following three weeks in storage at −20° C. In one aspect, the present invention provides a composition as described herein, wherein the level of manufactured bromoform in the composition is maintained following four weeks in storage at −20° C.

In one aspect, the present invention provides a composition as described herein, wherein the level of manufactured bromoform in the composition is maintained following one week in storage at 4° C. In one aspect, the present invention provides a composition as described herein, wherein the level of manufactured bromoform in the composition is maintained following two weeks in storage at 4° C. In one aspect, the present invention provides a composition as described herein, wherein the level of manufactured bromoform in the composition is maintained following three weeks in storage at 4° C. In one aspect, the present invention provides a composition as described herein, wherein the level of manufactured bromoform in the composition is maintained following four weeks in storage at 4° C.

In one aspect, the present invention provides a composition as described herein, wherein the level of manufactured bromoform in the composition is maintained following one week in storage at 25° C. In one aspect, the present invention provides a composition as described herein, wherein the level of manufactured bromoform in the composition is maintained following two weeks in storage at 25° C. In one aspect, the present invention provides a composition as described herein, wherein the level of manufactured bromoform in the composition is maintained following three weeks in storage at 25° C. In one aspect, the present invention provides a composition as described herein, wherein the level of manufactured bromoform in the composition is maintained following four weeks in storage at 25° C.

In one aspect, the present invention provides a composition as described herein, wherein the level of manufactured bromoform in the composition is maintained following one week in storage at 40° C. In one aspect, the present invention provides a composition as described herein, wherein the level of manufactured bromoform in the composition is maintained following two weeks in storage at 40° C. In one aspect, the present invention provides a composition as described herein, wherein the level of manufactured bromoform in the composition is maintained following three weeks in storage at 40° C. In one aspect, the present invention provides a composition as described herein, wherein the level of manufactured bromoform in the composition is maintained following four weeks in storage at 40° C.

In one aspect, the present invention provides a composition as described herein, wherein the level of manufactured bromoform in the composition is maintained following one week in open air conditions at 25° C. In one aspect, the present invention provides a composition as described herein, wherein the level of manufactured bromoform in the composition is maintained following two weeks storage in open air conditions at 25° C. In one aspect, the present invention provides a composition as described herein, wherein the level of manufactured bromoform in the composition is maintained following three weeks storage in open air conditions at 25° C. In one aspect, the present invention provides a composition as described herein, wherein the level of manufactured bromoform in the composition is maintained following four weeks in storage in open air conditions 25° C.

In one aspect, the present invention provides a composition as described herein, wherein the level of manufactured bromoform in the composition is maintained following one week in open air conditions at 40° C. In one aspect, the present invention provides a composition as described herein, wherein the level of manufactured bromoform in the composition is maintained following two weeks storage in open air conditions at 40° C. In one aspect, the present invention provides a composition as described herein, wherein the level of manufactured bromoform in the composition is maintained following three weeks storage in open air conditions at 40° C. In one aspect, the present invention provides a composition as described herein, wherein the level of manufactured bromoform in the composition is maintained following four weeks in storage in open air conditions 40° C.

In one aspect, the present invention provides a composition as described herein, wherein the level of manufactured bromoform in the composition is not reduced by more than 10, 20, 30, 40, 50, or 60% relative to the starting concentration of bromoform following one week in storage at −20° C. In one aspect, the present invention provides a composition as described herein, wherein the level of manufactured bromoform in the composition is not reduced by more than 10, 20, 30, 40, 50, or 60% relative to the starting concentration of bromoform following two weeks in storage at −20° C. In one aspect, the present invention provides a composition as described herein, wherein the level of manufactured bromoform in the composition is not reduced by more than 10, 20, 30, 40, 50, or 60% relative to the starting concentration of bromoform following three weeks in storage at −20° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage at −20° C.

In one aspect, the present invention provides a composition as described herein, wherein the level of manufactured bromoform in the composition is not reduced by more than 10, 20, 30, 40, 50, or 60% relative to the starting concentration of bromoform following one week in storage at 4° C. In one aspect, the present invention provides a composition as described herein, wherein the level of manufactured bromoform in the composition is not reduced by more than 10, 20, 30, 40, 50, or 60% relative to the starting concentration of bromoform following two weeks in storage at 4° C. In one aspect, the present invention provides a composition as described herein, wherein the level of manufactured bromoform in the composition is not reduced by more than 10, 20, 30, 40, 50, or 60% relative to the starting concentration of bromoform following three weeks in storage at 4° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage at 4° C.

In one aspect, the present invention provides a composition as described herein, wherein the level of manufactured bromoform in the composition is not reduced by more than 10, 20, 30, 40, 50, or 60% relative to the starting concentration of bromoform following one week in storage at 25° C. In one aspect, the present invention provides a composition as described herein, wherein the level of manufactured bromoform in the composition is not reduced by more than 10, 20, 30, 40, 50, or 60% relative to the starting concentration of bromoform following two weeks in storage at 25° C. In one aspect, the present invention provides a composition as described herein, wherein the level of manufactured bromoform in the composition is not reduced by more than 10, 20, 30, 40, 50, or 60% relative to the starting concentration of bromoform following three weeks in storage at 25° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage at 25° C.

In one aspect, the present invention provides a composition as described herein, wherein the level of manufactured bromoform in the composition is not reduced by more than 10, 20, 30, 40, 50, or 60% relative to the starting concentration of bromoform following one week in storage at 40° C. In one aspect, the present invention provides a composition as described herein, wherein the level of manufactured bromoform in the composition is not reduced by more than 10, 20, 30, 40, 50, or 60% relative to the starting concentration of bromoform following two weeks in storage at 40° C. In one aspect, the present invention provides a composition as described herein, wherein the level of manufactured bromoform in the composition is not reduced by more than 10, 20, 30, 40, 50, or 60% relative to the starting concentration of bromoform following three weeks in storage at 40° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage at 40° C.

In one aspect, the present invention provides a composition as described herein, wherein the level of manufactured bromoform in the composition is not reduced by more than 10, 20, 30, 40, 50, or 60% relative to the starting concentration of bromoform following one week in open air conditions at 25° C. In one aspect, the present invention provides a composition as described herein, wherein the level of manufactured bromoform in the composition is not reduced by more than 10, 20, 30, 40, 50, or 60% relative to the starting concentration of bromoform following two weeks storage in open air conditions at 25° C. In one aspect, the present invention provides a composition as described herein, wherein the level of manufactured bromoform in the composition is not reduced by more than 10, 20, 30, 40, 50, or 60% relative to the starting concentration of bromoform following three weeks storage in open air conditions at 25° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage in open air conditions 25° C.

In one aspect, the present invention provides a composition as described herein, wherein the level of manufactured bromoform in the composition is not reduced by more than 10, 20, 30, 40, 50, or 60% relative to the starting concentration of bromoform following one week in open air conditions at 40° C. In one aspect, the present invention provides a composition as described herein, wherein the level of manufactured bromoform in the composition is not reduced by more than 10, 20, 30, 40, 50, or 60% relative to the starting concentration of bromoform following two weeks storage in open air conditions at 40° C. In one aspect, the present invention provides a composition as described herein, wherein the level of manufactured bromoform in the composition is not reduced by more than 10, 20, 30, 40, 50, or 60% relative to the starting concentration of bromoform following three weeks storage in open air conditions at 40° C. In one aspect, the present invention provides a composition as described herein, wherein the level of manufactured bromoform in the composition is not reduced by more than 10, 20, 30, 40, 50, or 60% relative to the starting concentration of bromoform following four weeks in storage in open air conditions 40° C.

The present invention provides compositions that release bromoform as a function of time, as shown in Example 7, in one embodiment the present invention provides a sustained release composition comprising manufactured bromoform and a bromoform stabilising excipient.

Previous work has demonstrated that manufactured bromoform formulated in DMSO significantly inhibits methane production at doses of 5 μM, 10 μM and 25 μM, but at 1 μM, manufactured bromoform does not significantly inhibit methane production in in vitro fermentation. In contrast, the present inventors have surprisingly demonstrated that doses of manufactured bromoform of 0.04 mg per gram of OM of feed inhibit methane production from ruminal fermentation.

Importantly, FIG. 9 demonstrates that even lower doses of manufactured bromoform can be used in the methods and compositions described herein, and inhibit ruminal fermentation. For example, 0.01 mg manufactured bromoform per gram of feed substrate can be used in the compositions and methods of the present invention to inhibit methane production from ruminal fermentation.

In one embodiment, a composition according to the invention is administered at an amount based on actual individual animal intake (e.g. g/kg OM or g/kg DM intake). In contrast to Asparagopsis which is a natural product with a variable bromoform content, the present invention allows for compositions of defined manufactured bromoform content.

Accordingly, in one embodiment the present invention provides a method as described herein, wherein the composition is administered at a dose of at least 0.005, 0.01, 0.02, 0.03, 0.04, 0.05, 0.06, 0.07 or 0.08 mg of manufactured bromoform per gram of feed provided to the ruminant animal. In a preferred embodiment, the present invention provides a method as described herein, wherein the composition is administered at a dose of at least 0.03, 0.04, or 0.05, mg of manufactured bromoform per gram of feed provided to the ruminant animal.

In a preferred embodiment the present invention provides a method as described herein, wherein the composition is administered at a dose of at least 0.02, 0.03, 0.04, 0.05, 0.06, 0.07 or 0.08 mg of manufactured bromoform per gram of organic matter provided to the ruminant animal. In another embodiment wherein a composition according to the invention is administered at an amount based dry matter intake, the composition is administered at a dose of at least 0.005, 0.01, 0.02, 0.03, 0.04, 0.05, 0.06, 0.07 or 0.08 mg of manufactured bromoform per about 1.19 grams of dry matter provided to the ruminant animal.

In another embodiment the present invention provides a method as described herein, wherein the composition is made available at an amount to provide a dose of at least 0.005, 0.01, 0.02, 0.03, 0.04, 0.05, 0.06, 0.07 or 0.08 mg of bromoform per gram of organic matter provided to the ruminant animal. In another embodiment wherein a composition according to the invention is made available at an amount based dry matter intake, the composition is administered at a dose of at least 0.005, 0.01, 0.02, 0.03, 0.04, 0.05, 0.06, 0.07 or 0.08 mg of manufactured bromoform per about 1.19 grams of dry matter provided to the ruminant animal.

For example, if a ruminant animal consumes approximately 2.5-3% of its live weight of feed a day, a 400 kg ruminant animal may consume 10-12 kg of feed a day.

As discussed above, in preferred embodiments of the invention, an effective amount at manufactured bromoform is at least 0.005, 0.01, 0.02, 0.03, 0.04, 0.05, 0.06, 0.07 or 0.08 mg of manufactured bromoform per gram of organic matter provided to the ruminant animal, for example, per day, or 0.005, 0.01, 0.02, 0.03, 0.04, 0.05, 0.06, 0.07 or 0.08 mg of manufactured bromoform per about 1.19 grams of dry matter provided to the ruminant animal, for example, per day.

Therefore, if a 400 kg ruminant animal consumes about 10 kg of organic matter a day, an effective amount of manufactured bromoform in the composition is at least about 0.1, 0.2, 0.3, 0.4, 0.5, 0.6, 0.7, or 0.8 g of a composition as described herein per day. These doses are equivalent to 0.0005, 0.00075, 0.001, 0.00125, 0.0015, 0.00175 or 0.002 g of manufactured bromoform per kg body weight per day. Suitable doses can be calculated as described herein.

The effective amount can be administered to said ruminant animal in one or more doses.

The effective amount can also be administered to said ruminant animal in one or more doses on a daily basis.

In another preferred embodiment a method as defined herein before is provided, wherein the dosage of manufactured bromoform is within the range of 0.00025-0.0065 g/kg body weight per day, more preferably within the range of 0.0005-0.0065 g/kg body weight per day, most preferably 0.001-0.00275 g/kg body weight per day.

The dosages defined herein as the amount per kg body weight per day concern the average amount of the manufactured bromoform during a given period of treatment, e.g. during a week or a month of treatment. The compositions described herein may thus be provided or administered every day, every other day, every other two days, etc., without departing from the scope of the invention. Preferably though, the method comprises daily administration of a composition defined herein at defined dosages. Even more preferably the composition is provided or administered during feeding of the animal each time the animal is fed, in amounts yielding the above daily dosages.

The present method may comprise provision or administration of the composition in accordance with the described dosage regimens for a period of at least 5, 10, 25, 50, 100, 250 or 350 days.

In animal systems, including pasture-based systems, providing supplements to the system which allow for an effective dose to be administered to animals can be a problem using Asparagopsis biomass or Asparagopsis derived products. For example, if only a small proportion of a pasture-based animal's feed intake is feed supplement, then the dose of bromoform in the supplement is required to be high to provide an effective dose. Because high inclusion rates of Asparagopsis biomass or Asparagopsis derived products in feed supplements can make the supplement unpalatable to the animal, leading to an ineffective dose being consumed. The present invention allows for the preparation of feed supplements containing manufactured bromoform at levels to allow delivery of an effective dose in animal systems such as pasture-based/grazing systems.

The ability to prepare feed supplements containing manufactured bromoform at higher levels to allow delivery of an effective dose to animals in smaller amounts (e.g. feed volume) also allows for the delivery of an effective amount of the compositions described herein when the animals are at a feeding machine or during milking and the like.

Relative to Asparagopsis based or Asparagopsis derived oil products, the ability to preparate feed supplements containing manufactured bromoform at higher levels to allow delivery of an effective dose to animals in smaller amounts allows high levels of oil in an animal's feed to be avoided. The use of high levels of oil in animal feed can cause the oil containing feed to become partially liquefied, be unpalatable, to foul, and/or cause issues with feeding machines.

In another aspect, the present invention provides compositions for reducing total gas production and/or methane production and/or improving growth performance of a ruminant animal, wherein said composition comprises manufactured bromoform and a bromoform stabilising excipient

In another aspect, the present invention provides compositions when used for reducing total gas production and/or methane production and/or improving growth performance of a ruminant animal, wherein said composition comprises manufactured bromoform and a bromoform stabilising excipient

In one embodiment of the compositions for use, the bromoform stabilising excipient is an edible non-polar substance.

In one embodiment of the compositions for use, the edible non-polar substance is an edible oil.

In one embodiment of the compositions for use, the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In one embodiment of the compositions for use, the composition is formulated for provision to the ruminant animal at a dose of at least 0.005, 0.01, 0.02, 0.03, 0.04, 0.05, 0.06 or 0.08 mg of bromoform per gram of organic matter provided to the ruminant animal.

In one embodiment of the compositions for use, the composition is in a solid, a semi-solid or a liquid form.

In another aspect, the present invention provides a feed supplement when used for reducing total gas production and/or methane production in a ruminant animal, said supplement comprising an effective amount of a composition comprising manufactured bromoform and a bromoform stabilising excipient.

As used herein, the term “animal feed supplement” refers to a concentrated additive premix comprising the active ingredients, which premix or supplement may be added to an animal's feed or ration to form a supplemented feed in accordance with the present invention. The terms “animal feed premix,” “animal feed supplement,” and “animal feed additive” are generally considered to have similar or identical meanings and are generally considered interchangeable. Typically, the animal feed supplement of the present invention is in the form of a powder or compacted or granulated solid. In another embodiment, the animal feed supplement of the present invention is in the form of a mineral loose lick, wet lick, dry lick, pellets, water suspension, a liquid supplement (e.g. molasses blend) or lick block. In practice, livestock may typically be fed the animal feed supplement by adding it directly to the ration, e.g. as a so-called top-dress, or it may be used in the preparation or manufacture of products such as compounded animal feeds or a lick blocks, which will be described in more detail hereafter. The invention is not particularly limited in this respect. A supplement according to the invention is typically fed to an animal in an amount ranging from 16-3000 g or higher per animal per day.

The animal feed supplements of the present invention may comprise any further ingredient without departing from the scope of the invention. It may typically comprise well-known excipients that are necessary to prepare the desired product form and it may comprise further additives aimed at improving the quality of the feed and/or at improving the performance of the animal consuming the supplement. Suitable examples of such excipients include carriers or fillers, such as lactose, sucrose, mannitol, starch crystalline cellulose, sodium hydrogen carbonate, sodium chloride and the like and binders, such as gum Arabic, gum tragacanth, sodium alginate, starch, PVP and cellulose derivatives, etc. Examples of feed additives known to those skilled in the art include vitamins, amino acids and trace elements, digestibility enhancers and gut flora stabilizers and the like.

The term ‘compounded animal feed composition’ as used herein, means a composition which is suitable for use as an animal feed and which is blended from various natural or non-natural base or raw materials and/or additives. Hence, in particular, the term ‘compounded’ is used herein to distinguish the present animal feed compositions from any naturally occurring raw material. These blends or compounded feeds are formulated according to the specific requirements of the target animal. The main ingredients used in commercially prepared compounded feeds typically include wheat bran, rice bran, corn meal, cereal grains, such as barley, wheat, rye and oat, soybean meal, alfalfa meal, cottonseed meal, wheat powder and the like. A commercial compound feed will typically comprise no less than 15% of crude protein and no less than 70% digestible total nutrients, although the invention is not particularly limited in this respect.

Liquid, solid as well as semi-solid compounded animal feed compositions are encompassed within the scope of the present invention, solid and semi-solid forms being particularly preferred. These compositions are typically manufactured as meal type, pellets or crumbles. In practice, livestock may typically be fed a combination of compounded feed, such as that of the present invention, and silage or hay or the like. Typically, a compounded animal feed is fed in an amount within the range of 0.3-10 kg/animal/day. It is within the skills of the trained professional to determine proper amounts of these components to be included in the compounded animal feed, taking into account the type of animal and the circumstances under which it is held.

The compounded animal feed compositions of the invention may comprise any further feed additive typically used in the art. As is known by those skilled in the art, the term ‘feed additive’ in this context refers to products used in animal nutrition for purposes of improving the quality of feed and the quality of food from animal origin, or to improve the animals' performance, e.g. providing enhanced digestibility of the feed materials. Non-limiting examples include technological additives such as preservatives, antioxidants, emulsifiers, stabilising agents, acidity regulators, non-protein nitrogen (NPN) and silage additives; sensory additives, especially flavours and colorants; (further) nutritional additives, such as vitamins, amino acids and trace elements; and (further) zootechnical additives, such as digestibility enhancers and gut flora stabilizers. As will be clear to those skilled in the art, the present compounded animal feed compositions can comprise any further ingredient or additive, without departing from the scope of the invention.

In a further aspect, the invention provides a lick stone or lick block comprising the supplement of the invention. As is known to those skilled in the art such lick stones or blocks are particularly convenient for feeding mineral supplements (as well as proteins and carbohydrates) to ruminants grazing either or both natural and cultivated pastures. Such lick blocks or lick stones in accordance with the present invention typically comprise, in addition to the composition of the invention, various types of binders, e.g. cements, gypsum, lime, calcium phosphate, carbonate, and/or gelatin; and optionally further additives such as vitamins, trace elements, mineral salts, sensory additives, etc.

In one embodiment of the feed supplement for use, the feed supplement is formulated to provide a dose of at least 0.005, 0.01, 0.02, 0.03, 0.04, 0.05, 0.06 or 0.08 mg of manufactured bromoform per gram of organic matter provided to the ruminant animal.

In another embodiment of the feed supplement for use, the feed supplement is formulated to provide a dose of at least 0.005, 0.01, 0.02, 0.03, 0.04, 0.05, 0.06 or 0.08 mg of manufactured bromoform per gram of feed substrate provided to the ruminant animal.

In one embodiment of the feed supplement for use, the feed supplement further comprises feed additives

In one embodiment of the feed supplement for use, the ruminant animal is selected from the members of the Ruminantia and Tylopoda suborders.

In one embodiment of the feed supplement for use, the ruminant animal is cattle or sheep.

In one embodiment of the feed supplement for use, the bromoform stabilising excipient is an edible non-polar substance.

In one embodiment of the feed supplement for use, the edible non-polar substance is an edible oil.

In one embodiment of the feed supplement for use, the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In one embodiment of the feed supplement for use, the composition is in a solid, a semi-solid or a liquid form.

Methods of preparing methane reducing ruminant animal feed are described herein.

Accordingly, in one aspect the present invention provides a method of producing a methane reducing ruminant animal feed, comprising mixing a ruminant animal feed with a feed supplement comprising an effective amount of a composition comprising manufactured bromoform and a bromoform stabilising excipient.

In one embodiment of the method of producing a methane reducing ruminant animal feed, the animal feed comprises a dose of at least 0.005, 0.01, 0.02, 0.03, 0.04, 0.05, 0.06 or 0.08 mg of bromoform per gram of organic matter of the ruminant animal feed.

In another aspect, the present invention provides an animal feed when used for reducing total gas production and/or methane production in a ruminant animal, said supplement comprising an effective amount of a composition comprising manufactured bromoform and a bromoform stabilising excipient and a ruminant animal feed.

In one embodiment of the animal feed when used for reducing total gas production and/or methane production in a ruminant animal, the feed supplement is formulated to provide a dose of at least 0.005, 0.01, 0.02, 0.03, 0.04, 0.05, 0.06 or 0.08 mg of bromoform per gram of the organic matter of the ruminant animal feed.

In one embodiment of the animal feed when used for reducing total gas production and/or methane production in a ruminant animal, the ruminant animal is selected from the members of the Ruminantia and Tylopoda suborders.

In one embodiment of the animal feed when used for reducing total gas production and/or methane production in a ruminant animal, the ruminant animal is cattle or sheep.

In one embodiment of the animal feed when used for reducing total gas production and/or methane production in a ruminant animal, the bromoform stabilising excipient is an edible non-polar substance.

In one embodiment of the animal feed when used for reducing total gas production and/or methane production in a ruminant animal, the edible non-polar substance is an edible oil.

In one embodiment of the animal feed when used for reducing total gas production and/or methane production in a ruminant animal, the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

In one embodiment of the animal feed when used for reducing total gas production and/or methane production in a ruminant animal, the composition is in a solid, a semi-solid or a liquid form.

The invention will now be further described by way of Examples, which are meant to serve to assist one of ordinary skill in the art in carrying out the invention and are not intended in any way to limit the scope of the invention.

### EXAMPLES

### Example 1: Materials and Methods

### Preparation of Feed Substrate and Oil Products

Rhodes grass hay was used as the primary feed substrate and was first dried and ground to 1 mm, then supplied to each vessel at 1.25 g DM (1.0 g OM).

The Asparagopsis seaweed was prepared by FutureFeed Pty Ltd, Townsville, Queensland, Australia which was sourced from Middle Reef at Magnetic Island, Queensland, Australia. Approximately 20 kg of fresh harvested Asparagopsis was first rinsed in seawater for two minutes, spun dry three times [1-2 minutes each time] then added to 20 L of refined canola oil for 20 days. An additional collection of fresh Asparagopsis was collected from the same area and the process was repeated as above with the same 20 L of refined canola oil to achieve the final CHBr₃ concentration of 3.60 mg/g (Table 1) in the composition comprising Asparagopsis biomass in oil (“Asp-Oil”).

A composition comprising manufactured bromoform in oil (synthetic CHBr₃ oil; “Syn-Oil”) was produced by FutureFeed Pty Ltd using the same refined canola oil that was used to make the Asp-Oil. Synthetic CHBr₃ stabilized in amylene (Sigma-Aldrich, product no: 36972) was dissolved and homogenised into the canola oil by repeated inversion and left to stabilise overnight before analysis. The oil was then volumetrically diluted to achieve comparable CHBr₃ concentrations between the Asp-Oil and Syn-Oil (Table 1).

A subsample of freeze-dried Asparagopsis previously proven to reduce enteric CH₄ emissions both in vivo and in vitro (Roque et al 2019, Kinley et al 2020, Roque et al 2021) was added as a positive control group during Experiment 2.

Bromoform concentrations for the Asp-Oil was 3.60±0.05 mg/g DW, Syn-Oil was 3.69±0.05 mg/g DW, and freeze-dried Asparagopsis was 7.7 mg/g DW. Bromoform concentrations confirmed using an improved analytical protocol as previously described by Paul et al (2006). et al

TABLE 1
Dry matter, organic matter, and bromoform content of the
Rhodes grass substrate, freeze dried Asparagopsis &
Asparagopsis oil, and Synthetic bromoform oil.

| | | Freeze | | |
|---|---|---|---|---|
| | Rhodes | Dried | Oil + | Oil + |
| Composition | grass | Asparagopsis | Asparagopsis | Bromoform |
| Dry matter | 92.4 | 94.5 | 100 | 100 |
| (% As-fed) | | | | |
| Organic matter | 88.2 | 51.3 | 100 | 100 |
| (% DM) | | | | |
| Bromoform | — | 7.7 | 3.60 | 3.69 |
| (mg/g DM) | | | | |

### Donor Animals and In Vitro Preparation

The donor animals were maintained at the Commonwealth Scientific and Industrial Research Organization (CSIRO) Lansdown Research Station near Townsville, Queensland, Australia (coordinates; 19°39′27.000″S, 146° 50′04.60″ E) according to current guidelines (NHMRC 2013) and approved by the Commonwealth Scientific and Industrial Research Organisation (CSIRO) animal ethics committee (Ethical Clearance Certificate 2018-37). Rumen fluid (RF) was collected from four fistulated Brahman steers fitted with 10-cm Bar Diamond (Parma, OH, USA) rumen cannulas. All four steers were allowed to graze rangeland and supplemented with Rhodes grass hay ad libitum before RF collection, which was extracted two hours after morning feeding then placed into a 1-L pre-warmed, insulated thermos and transported back to the lab. All RF was pooled, filtered through a 0.5 mm sieve, then combined with an artificial saliva buffer (Goering and van Soest 1970) at a ratio of 1 part RF and 4 parts buffer. Approximately 125 mL of the RF buffer mixture and 1.25 g DM of substrate was allocated to 250 mL Ankom incubation bottles, headspace purged with N2, then sealed with the Ankom RF1 gas production module (Macedon, New York, United States). Incubation bottles were placed into Ratek OM11 dry incubators (Boronia, Victoria, Australia) and maintained at a constant temperature of 39° C. and oscillation speed of 85 RPM.

### Sampling and Analysis

### Total Gas, CH₄, and Hydrogen Production

Total gas production (TGP), CH₄, hydrogen and carbon dioxide production were determined using Ankom RF gas production technology (Macedon, NY, USA). Briefly, the Ankom RF modules were set to maximum pressure of 3 psi which when exceeded would vent for 250 milliseconds. Live interval (LI) was set at 60 seconds monitoring of gas production each measurement was corrected for ambient pressure change via ambient Ankom RF monitors. The recording interval (RI) was set to 20 minutes thus LI cumulative pressure change was recorded at each RI as 20 min contributions to the cumulative pressure change over the duration of the fermentation (24, 48, or 72 h). Total cumulative pressure change was converted to TGP using the natural gas law and corrected for absolute volume of individual fermentation bottles. In vitro CH₄ production was determined by analysis of headspace gas relative to TGP while assuming constant homogeneity of bottle headspace. At termination of each incubation period headspace gas samples from individual fermentation bottles were collected through the Ankom RF module vent tube into 10-mL Labco Exetainer vacuum vials (Lampeter, Great Britain). Gas samples were analysed by gas chromatography (GC) on a Shimadzu GC-2014 (Kyoto, Japan) equipped with a Restek (Bellefonte, PA, USA) ShinCarbon ST 100/120 column (2 m· 1 mm· micropacked) with a flame ionisation detector (FID). Column temperature was set to 150° C., injector at 240° C., and FID at 380° C. Ultra high purity N2 was the carrier gas at 25 mL/min and total injection volume was 250 μL.

### Apparent Digestibility

After the fermentation bottles were sacrificed [either at 24, 48, or 72-hr] they were chilled in a −20° C. freezer, to cease bacterial activity, then the rumen liquor was vacuum filtered through 50 mL Gooch sintered disc glass filtering crucibles, porosity 1, (GLASSCO; Haryana, India) with a 0.5-cm layer of sand filtration aid. The crucible containing the digested rumen liquor residues was then oven dried at 105° C. until constant weight was achieved then was subtracted from total DM added to the bottle to determine in vitro apparent digestibility of substrate dry matter (IVD-DM). The resultant dried residues then ashed at 530° C. to determine apparent digestibility of substrate organic matter (IVD-OM).

### Example 2: A Composition Comprising Bromoform and a Bromoform Stabilising Excipient Reduces Methane Production in Ruminal Fermentation

### In Vitro Set Up and Experimental Design

Asparagopsis oil (“Asp-oil”) was tested at four levels of inclusion [0.08 mg, 0.11 mg, 0.15 mg, and 0.18 mg] and Synthetic CHBr₃ oil (Syn-oil) was tested at six inclusion levels [0.08 mg, 0.11 mg, 0.15 mg, 0.18 mg, 0.22 mg, and 0.26 mg per gram of organic matter (OM of feed)] along with four control treatments that varied in canola oil inclusion to match levels 1, 2, 4, and 6 of the Asp-Oil and Syn-Oil products. Unsaturated oils can be a CH₄ mitigating feed ingredient on its own (Grainger & Beauchemin, 2011) therefore it was necessary to test a range of inclusions for oil as well. All experimental treatments [14 total] were tested in duplicate over 2 fermentation timepoints: 24 and 48 hours conducted during the in vitro incubation period. Data collected for each treatment, fermentation timepoint, and incubations were then combined and analysed. Note that CH₄ production on feed-digested basis could not be reported here because IVDDM was not measured in Example 2 which was designed for demonstration of CH₄ production only. However, all fermentations received the same Rhodes grass and canola oil feed substrate at equivalent levels. Reporting on feed-digested basis would have little effect on the shape of the as-fed basis of FIG. 1.

### Results

FIG. 1 demonstrates that the Asp-Oil and the composition comprising bromoform and a bromoform stabilising excipient (Syn-Oil) were both demonstrated to be highly antimethanogenic at equivalent levels of CHBr₃ delivery in vitro.

Hydrogen (H₂) emission, also shown in FIG. 1, is normally non-existent as shown by the absence of hydrogen concentrations in the controls as H₂ is taken up by methanogens to produce CH₄ which as indicated in the formula contains four hydrogen (H⁺) atoms and acts as a hydrogen sink. In concord with CH₄ mitigation, H₂ concentrations increase across all CHBr₃ inclusion levels for both Asp-Oil and Syn-Oil products with the exception of Syn-Oil 0.08 mg at the 48-hour fermentation timepoint due to reduced CH₄ mitigation efficacy at 48 hours.

Total gas production (TGP) is also included in FIG. 1 to demonstrate maintenance of microbial activity within all treatments including those with CHBr₃ content. A slight drop in TGP can be expected due to the proportion of CH₄ that makes up TGP in the control treatments.

These results demonstrate that the Asp-Oil inhibits methane production from rumen fermentation, and that a composition comprising bromoform and a bromoform stabilising excipient is able to reduce methane production from rumen fermentation in the absence of the other secondary metabolites (and other components) of Asparagopsis.

### Example 3: Dose-Dependent Responses of Oil Products on Gas Production and Digestibility During a 72-Hour Period

### In Vitro Set Up and Experimental Design

Two oil products were used [substrate+Asp-Oil or Syn-Oil] along with one negative control (C1-) [substrate+0.02 g canola oil], one positive control (C2+) [substrate+0.01 g freeze-dried Asparagopsis+0.02 g canola oil], and one blank [no substrate or treatment, rumen fluid only].

Three levels of Asp-Oil or Syn-Oil inclusions were tested based CHBr₃ content and weight of material and referred to as low [0.04 mg], medium [0.07 mg], and high [0.11 mg] inclusion levels. All experimental treatments [9 total] were tested in duplicate over 3 fermentation timepoints: 24, 48, and 72 hours which were conducted during one in vitro incubation period. Data collected for each treatment, fermentation timepoint, and incubations were then combined and analysed for timeseries results.

### Results and Discussion

The design of Example 3 was based on Asp-Oil and Syn-Oil inclusion levels as informed by Example 2 to provide a minimum effective inclusion level that consistently reduced CH₄ over a 72-hour period. Based on the results of Example 2, the present inventors hypothesized that a CHBr₃ inclusion level of 0.04 mg for both Asp-Oil and Syn-Oil would inhibit methane production, but to a lesser degree than higher doses of bromoform.

The CH₄ results are presented in FIG. 2A demonstrate that 0.07 mg/g inclusion level showed a stronger CH₄ mitigation capability than expected based on the data of Example 2. The 0.11 mg inclusion for both Asp-Oil and Syn-Oil completely inhibited CH₄ production throughout the duration of the study.

This data indicates that a composition comprising bromoform and a bromoform stabilising excipient is able to reduce methane production in the absence of the other secondary metabolites (and other components) of Asparagopsis.

At 48 hours, the Asparagopsis-derived composition comprising Asparagopsis biomass extracted into a bromoform stabilising excipient (Asp-Oil), which comprises other antimethanogenic secondary metabolites (and other components) of Asparagopsis, was able to reduce methane production to a greater extent than Syn-Oil, which does not comprise other antimethanogenic secondary metabolites (and other components) of Asparagopsis.

In addition to the oil treatments, a positive control of freeze-dried (FD) Asparagopsis was included in this study at a CHBr₃ inclusion of 0.07 mg and worked similarly to the mid (0.07 mg), and high (0.11 mg) oil inclusion levels.

FIG. 2 also demonstrates hydrogen significantly increases with the downregulation of CH₄ production then is consistently reduced with when CH₄ production increases again). Total gas production (TGP) is reduced universally accordingly with reduction in the CH₄ component of the TGP, thus TGP is demonstrated as equivalently lower than the Controls without FD-Asp, Asp-Oil, or Syn-Oil. Additional to this, in vitro dry matter digestibility (IVDMD) was measured to ensure no negative impact on dietary digestibility due to inclusion of the anti-methanogenic products and appears to be unaffected across treatment groups (bottom right, FIG. 2). Maintenance of TGP and IVDMD are strong indications that rumen microbial activity is stable with the inclusion of CH₄ mitigating oil products.

These results demonstrate that the Asp-Oil inhibits methane production from rumen fermentation, and that a composition comprising bromoform and a bromoform stabilising excipient is able to reduce methane production from rumen fermentation in the absence of the other secondary metabolites (and other components) of Asparagopsis.

### Example 4: Dose-Dependent Responses of Oil Products on In Vitro Gas Production and Digestibility During a 72 Hour Period

To compare enteric methane mitigation and effects on enteric fermentation (e.g. in vitro digested dry matter (IVDDM) and rumen volatile fatty acid production (VFA) as fermentation markers), the present inventors examined the responses in in vitro fermentation.

### Methods

Two oil products were used [substrate+Canola Oil or Asp-Oil] along with one negative control (CON) [substrate+0.02 g canola oil], one positive control (FD-Asp) [substrate+freeze dried Asparagopsis fed at 0.05 mg CHBr₃/g substrate] and one blank [no substrate or treatment, rumen fluid only]. Three levels of Canola Oil and Asp-Oil inclusions were tested based CHBr₃ content and weight of material and referred to as 0.03 [mg/g substrate], 0.05 [mg/g substrate], and 0.08 [mg/g substrate] inclusion levels. All experimental treatments [9 total] were tested in duplicate over 3 fermentation periods: 24, 48, and 72 hours which were replicated during three in vitro incubation periods. Data collected for each treatment, fermentation period, and incubations were then combined and analysed for timeseries results.

The Asparagopsis seaweed was prepared by FutureFeed Pty Ltd, Townsville, Queensland, Australia which was sourced off the coast of Magnetic Island, Queensland, Australia. Approximately 20 kg of fresh harvested Asparagopsis was first rinsed in seawater for two minutes, spun dry three times [1-2 minutes each time] then added to 20 L of refined canola oil for 20 days. After steeping for 3 weeks, the Asparagopsis biomass was removed and subsequently an additional 20 kg of fresh Asparagopsis, collected from the same area, was added to the canola oil and the process was repeated as above to achieve the final CHBr₃ concentration [3.03 mg/g±0.03].

Manufactured synthetic CHBr₃ of 98% purity and stabilized with amylene (Sigma-Aldrich, product no: 36972) was dissolved and homogenised into the canola oil by repeated inversion and left to stabilise overnight before analysis. The oil was then volumetrically diluted to achieve comparable CHBr₃ concentrations as the Asparagopsis oil.

Total gas production (TGP), CH₄, hydrogen (H₂) and carbon dioxide (CO₂) production were determined using Ankom RF gas production technology (Macedon, NY, USA) as described previously. Briefly, total cumulative pressure change was converted to TGP using the natural gas law and corrected for absolute volume of individual fermentation bottles. At termination of each incubation period headspace gas samples from individual fermentation bottles were collected through the Ankom RF module vent tube into 10-mL Labco Exetainer vacuum vials (Lampeter, Great Britain) and used to determine in vitro CH₄, CO₂, and H₂ production, while assuming constant homogeneity of bottle headspace.

Additionally, IVDDM and VFA were determined. In brief, after the fermentation bottles were sacrificed [either at 24, 48, or 72-hr] they were chilled in a −20° C. freezer, to cease bacterial activity, then the rumen liquor was vacuum filtered through sintered glass disc filtering crucibles with a 0.5-cm layer of sand filtration aid. The crucible containing the digested rumen liquor residues was then oven dried at 105° C. until constant weight was achieved then was subtracted from total DM added to the bottle to determine IVDDM. A 1.5 mL sample of rumen fluid was collected from the 72-hr fermentation bottles for a determination of total VFA (tVFA) as well as acetate, propionate, and butyrate production.

### Results

The design of Example 4 was based on Asp-Oil and Canola Oil inclusion levels as informed by in vitro Experiments-1 and 2 to provide a range of effective inclusion levels (i.e dose response curve) that consistently reduced CH₄ over a 72 h fermentation period.

The CH₄ results are presented in FIG. 3, which demonstrates that Asparagopsis biomass extracted into oil (“Asp-Oil”), and manufactured bromoform (“Canola Oil”) in oil as a stabilising excipient performed in in a similar manner.

The 0.05 inclusion level showed an even stronger CH₄ mitigation capability than expected for the Asp-Oil, Canola Oil, and freeze dried Asparagopsis biomass (“FD-Asp”) during the 24 h fermentation period however, as expected, a small universal reduction in effectiveness appeared over 48 and 72 h of fermentation. Additionally, during the 48 and 72 h periods, FD-Asp appears to mitigate CH₄ more effectively than Asp-Oil and Canola Oil at the same inclusion level of 0.05 [mg/g substrate]. As expected based on Examples 1 to 3, the 0.08 inclusion for both Asp-Oil and Canola Oil completely inhibited CH₄ production throughout the duration of the study. This indicates stability of mitigation efficacy when CHBr₃ is provided within the optimal range.

As demonstrated in previous in vitro and in vivo studies, FIG. 4 shows H₂ increases with the downregulation of CH₄ production then is consistently reduced when CH₄ production resumes (FIG. 4). No negative impacts to fermentation were observed on IVDDM and tVFA production (FIGS. 5A & B) due to the inclusion of Asp-Oil or Canola Oil and appears to be unaffected across inclusion groups. Additional to this, reduction of Acetate and increased production of Propionate and Butyrate occurred at equal proportions between Asp-Oil and Canola Oil at the respective inclusion rates, providing evidence that Asp-Oil and Canola Oil work similarly, in an in vitro fermentation setting.

These results demonstrate that the Asp-Oil inhibits methane production from rumen fermentation, and that a composition comprising bromoform and a bromoform stabilising excipient is able to reduce methane production from rumen fermentation in the absence of the other secondary metabolites (and other components) of Asparagopsis.

### Example 5: Compositions Comprising Manufactured Synthetic Bromoform Stabilised in Corn Oil, Molasses, Cyclodextrin, and Water Inhibit Methane Production in Enteric Fermentation

To examine the ability of manufactured bromoform to be stabilised in different excipients and to inhibit methane production in enteric fermentation, manufactured CHBr₃ was stabilized in an edible oil (corn oil) a solid carbohydrate (Cyclodextrin), in a liquid form (Water), or in a semi-liquid carbohydrate (Molasses),

### Methods

Four CHBr₃ stabilizing excipients were used [substrate+Corn Oil, Molasses, Cyclodextrin, or Water] along with one negative control (“CON”) [substrate only], and one blank [no substrate or treatment, rumen fluid only]. Three inclusion levels of each excipient were tested based CHBr₃ content and weight of material and referred to as 0.01 [mg/g substrate], 0.04 [mg/g substrate], and 0.07 [mg/g substrate] inclusion levels. The experiment consisted of three in vitro incubation replications (n=3) including each of the CHBr₃ products experimental treatments [14 total] which were tested in duplicate within each replication over 3 fermentation periods: 24, 48, and 72 hours. Data collected for each treatment, fermentation period, and incubations were then combined and analysed for time series results.

Manufactured synthetic CHBr₃, 98% purity, and stabilized with 2-methyl-2-butene (Sigma-Aldrich, product no: 241032) was used for the preparation of the four excipients. The Corn Oil, Molasses and Water were made with similar protocols as per the Canola Oil in Example 4. Briefly, CHBr₃ was dissolved and homogenised into either retail corn oil, molasses, or deionised water and homogenised by repeated inversion and left to stabilise overnight prior to achieve a concentration of 3.0±0.10 mg/g. Cyclodextrin was prepared as described previously (patent WO 96/14062, PCT AU95/00733). Briefly, CHBr₃ was added to α-cyclodextrin (Sigma-Aldrich, product no: 779008) aqueous solution and mixed overnight. The solid was then separated, washed, then dried. The CHBr₃ concentration of the dried powder was determined and then diluted with additional α-cyclodextrin to adjust the CHBr₃ concentration to 3.0±0.10 mg/g. TGP, CH₄, H₂ and CO₂ production, IVDDM, and VFA were determined using the protocol as described in Example 4.

### Results

The design of Example 5 used inclusion levels as informed by an initial exploratory experiment (data not shown, similar experimental design as Example 1) in order to provide a range of effective inclusion levels (i.e dose response curve) for each CHBr₃ stabilizing excipient used over a 72 h fermentation period.

The CH₄ results are presented in FIG. 6, and demonstrate that manufactured bromoform can be stabilised in an edible oil other than canola (corn oil), solid excipient (Cyclodextrin), in a liquid excipient (Water), or in a semi-liquid excipient ((Molasses).

The level of efficacy from the Molasses and Water excipients at each inclusion level and over the 24, 48, and 72 h fermentation periods, which showed a similar CH₄ reduction to that of the Corn Oil and Cyclodextrin, was unexpected. This is of particular interest because CHBr₃ does not have a strong retention in water-based compounds (see Example 6), thus it was expected that there would be a decline in efficacy over the course of the 4 incubation periods.

At 24 h of fermentation, there appears to be increases in H₂ production across all excipients at the 0.04 and 0.07 inclusion rates, however due to the large variability in H₂ measurements, the results no statistical significance was found (FIG. 7). During the 48 h period, Cyclodextrin and Water resulted in significantly increased H₂ for the 0.04 and 0.07 inclusion rates whereas the Molasses and Corn Oil only showed significant increases at the 0.07 inclusion rate. During the 72 h period, Cyclodextrin, Water, and Molasses resulted in significantly increased H₂ for the 0.07 inclusion rate.

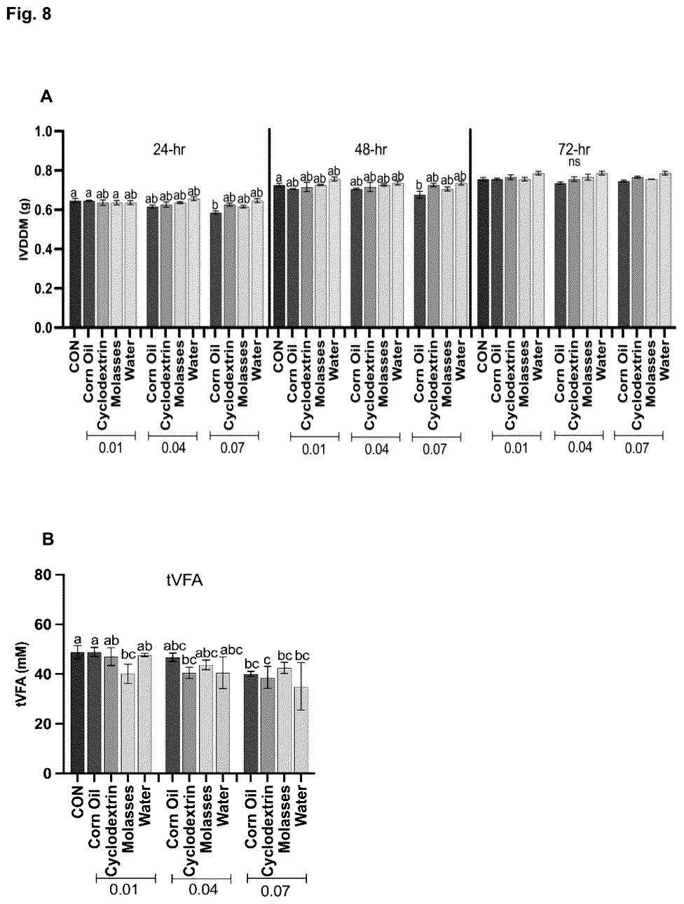

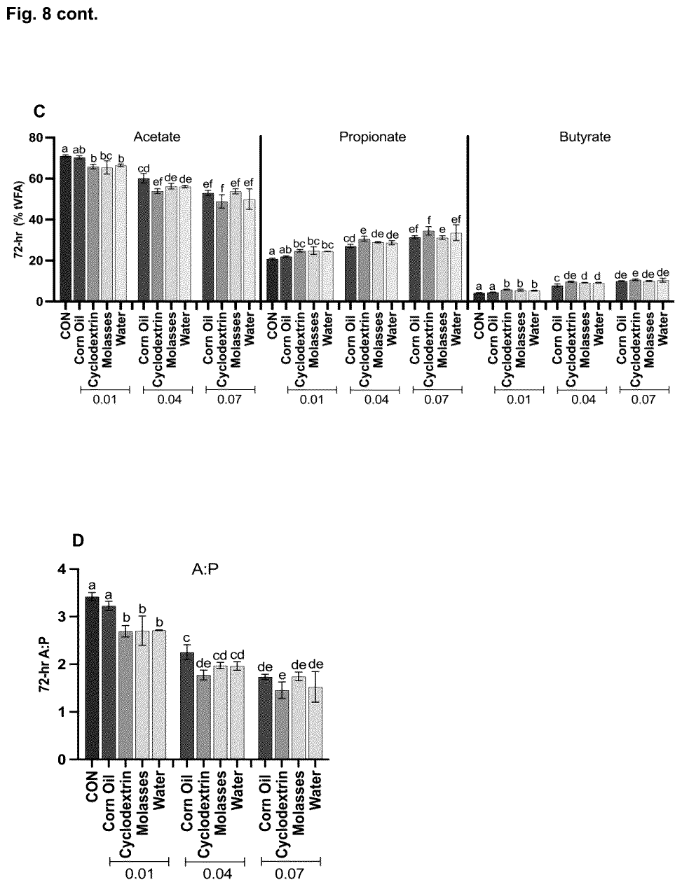

Corn Oil at the 0.07 inclusion demonstrated a potential decrease in IVDDM compared to control during 24 and 48 h of fermentation, however there were no differences in the 72 h period (FIG. 8A). No other excipient at any inclusion rate or period showed an effect on IVDDM. tVFA production (FIG. 8 B) at 72 h of fermentation seems to be impacted by the inclusion the four excipients, with the largest decrease in tVFAs shown in the highest inclusion rate of 0.07 [mg/g substrate]. Without wishing to be bound by theory, the present inventors propose this trend could be a reflection of the decreased production of Acetate, which is typically the most abundant VFA in the rumen, however is downregulated when CH₄ is inhibited. Reduction of Acetate, and increased production of Propionate, and Butyrate (FIG. 8 C) from all excipients show an individual significant influence on the production of the individual VFAs, at a particular inclusion rate, with Cyclodextrin being the most influential. As a result, the same trend holds true for the A:P ratio (FIG. 8 D). This is likely due to the high methane reduction efficacy of Cyclodextrin at each inclusion rate compared to the Corn Oil, Molasses, and Water.

FIG. 9 presents a summary of the data of Examples 4 and 5 in relation to the different CHBr₃ stabilizing excipients (grey bars) and the comparative Asparagopsis products (black bars) and a variety of different inclusion rates over 24, 48, and 72 h of fermentation. This aggregation of data is particularly interesting in that it illustrates the CHBr₃ inclusion at between 0.03-0.05 mg CHBr₃ per gram of feed substrate [30-50 mg/kg] results in complete inhibition of enteric CH₄.

These results demonstrate that the Asp-Oil inhibits methane production from rumen fermentation, and that compositions comprising bromoform and solid, liquid and semi-solid bromoform stabilising excipients are able to reduce methane production from rumen fermentation in the absence of the other secondary metabolites (and other components) of Asparagopsis.

These results also demonstrate that the compositions and methods of the present invention can be used to inhibit methane production at significantly lower doses of bromoform than has been demonstrated previously.

### Example 6: Asparagopsis Biomass Extracted into Oil (“Asp-Oil”), and Manufactured Bromoform Stabilised in Oil (“Canola Oil”) Inhibit Methane Production In Vivo

To examine the ability of stabilised compositions to inhibit methane production in vivo, the present inventors provided animals with a feed supplement comprising Asparagopsis biomass extracted into oil (“Asp-Oil”), or manufactured bromoform stabilised in canola oil (“Canola Oil”)

### Methods

A total of 60 early lactation dairy cows were randomly assigned to one of 10 treatment groups: Asp-Oil [inclusion level 0, 1, 2, 3, or 4] or Canola Oil [inclusion level 0, 1, 2, 3, or 4]. Inclusion levels were initially set to achieve 6, 12, 18, and 20 mg CHBr₃/kg DMI (on a daily basis) based on previous experimental research using Asp-Oil in lactating dairy cows. Asp-Oil and Canola Oil were mixed into the dairy cow's grain supplement that was offered twice daily at milking as two pulse doses separate from the measured primary daily feed intake occurring after milking. The initial introduction to the Asp-Oil and Canola Oil was done gradually over a 15-day adaptation period, consisting of 2-day incremental ramp ups of the respective oils into their feed supplement. This standard precaution minimizes the potential for ruminal upset and refusal to eat the supplement. After this period, cows were fed their respective diets and treatments for a further 21 days, then CH₄ measurements were taken using a sulphur hexafluoride (SF₆) measuring technique. In brief, an inert permeation tube is inserted in the rumen which constantly and consistently emits a standardized tracer of SF₆. Dilution of the tracer by background and rumen and respiratory gases provides for accurate partitioning of gas components. Representative respiration and eructated gases are collected in 24 h sampling sets for 5 consecutive days. Continuous sampling of individual cows during the collection is achieved using vacuum canisters drawing gases through the sampling tube from the sampling point above the cow's nostrils. Background gases are accounted through collection with a second canister drawing gases from the cow's flank.

### Results

FIG. 10 illustrates CH₄ reduction results prior to statistical analysis and reduction of CH₄ production [grams CH₄/day] compared to Control (no CHBr₃) was demonstrated up to 32% for Canola Oil and 38% for Asp-Oil at the high inclusion level of CHBr₃ [20 mg CHBr₃/kg DMI]. When CH₄ is standardized on a per kg dry matter intake (DMI) bases (CH₄ Yield), Canola Oil resulted in up to 28% reduction and Asp-Oil resulted in a 36% reduction.

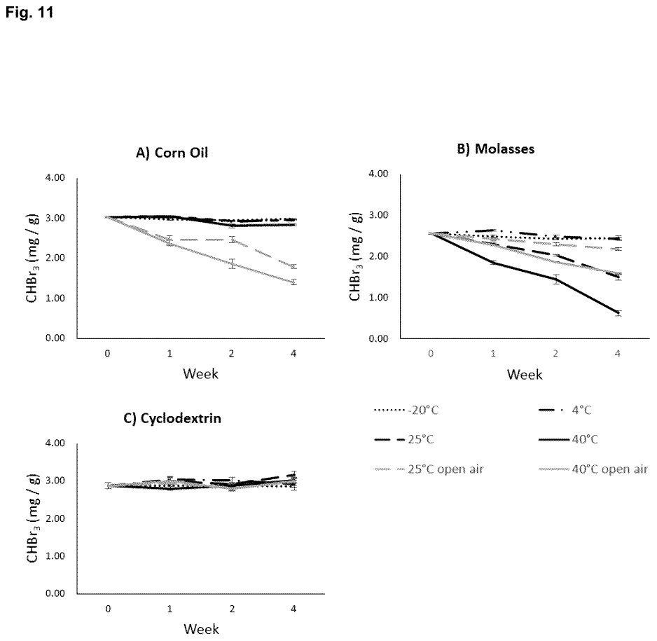

The range of CH₄ reductions is consistent with CH₄ reductions and respective inclusion rates demonstrated in vitro after 24 h of fermentation and illustrated in FIG. 11.

These results demonstrate that compositions comprising manufactured bromoform and a stabilising excipient that inhibit methane production from fermentation in vitro are able to inhibit methane production in vivo. This contrasts with the work of Stefenoni et al. which indicates manufactured bromoform is unable to inhibit methanogenesis.

These results also demonstrate that the Asp-Oil inhibits methane production from rumen fermentation, and that a composition comprising bromoform and a bromoform stabilising excipient similarly reduces methane production in vivo, in the absence of the other anti-methanogenic metabolites (and other components) of Asparagopsis.

### Example 7: Shelf-Life/Stability of Compositions Comprising Manufactured Bromoform and Bromoform Stabilising Excipients

To investigate the retention of manufactured synthetic bromoform (CHBr3) in the stabilised compositions, bromoform levels in the following excipients: corn oil, molasses, and cyclodextrin were monitored over a 4 week period under different storage temperatures and air exposure conditions.

### Methods

Asparagopsis products (FD-Asp and Asp-Oil) were investigated for their ability to retain CHBr3 content over a range of different temperatures, exposure to UV lighting, and exposure to open air, which was used as a framework for testing the shelf life stability of manufactured synthetic CHBr3 in the current study. Preparation for the three excipients [Corn Oil, Molasses, and Cyclodextrin] is described in Example 5. The starting concentration of CHBr3 in each excipient was between 2.5-3.0 mg/g (mg CHBr3/g excipient).

Amber test vials were used to store individual excipient products at four gradient temperatures [−20° C., 4° C., 25° C. or 40° C.] either sealed airtight and at temperatures 25° C. or 40° C., or unsealed and exposed to open air [6 treatment groups for each excipient]. Triplicate samples from each excipient, temperature, and air exposure were sacrificed at the start of the project (Week 0), then on Weeks 1, 2, and 4 for the determination of CHBr3 content which was analysed using GC-MS following the method described in Tan et al. 2022. The weight of open-air vials was measured at each measurement period for determination of moisture loss. Molasses samples exposed to open air lost a significant amount of moisture over the 4 weeks and subsequently the CHBr3 result was adjusted to account for the weight loss within the sample. The mean and standard error of each storage temperature/condition after each storage period was calculated and the effect of each treatment over time was analysed by one-way ANOVA using Tukey's Honest Significant Difference (HSD). Results were declared significant at P<0.5.

### Results

The CHBr₃ content in Corn Oil is stable when stored at −20° C., 4° C. and 25° C., over 4 weeks (FIG. 11, Table 2). However, when stored at 40° C., the concentration of CHBr₃ decreased after 2 weeks by 6.3%. Extending the duration of exposure would contribute to better understanding of prolonged storage impact on retention of CHBr₃ in Corn Oil when stored at 40° C. in airtight vials. When exposed to open air conditions CHBr₃ decreased significantly each week at 25° C. and 40° C., with an overall loss of 41.1% and 53.6%, respectively, over the 4 week period.

The Molasses CHBr₃ content appears to be stable at both −20° C. and 4° C. in airtight containers. However, at higher temperatures, the CHBr₃ content appears to be less stable in Molasses, compared to Canola or Corn oils. CHBr₃ in Molasses decreased significantly at 25° C. and 40° C. in both airtight and open-air conditions. In the airtight vials, at 25° C. and 40° C., CHBr₃ content decreased by 41.4% and 75.4%, respectively after 4 weeks of storage. Surprisingly, when exposed to open air during storage the CHBr₃ content of molasses more successful at retaining the CHBr₃ compared to the airtight counterpart with CHBr₃ losses of only 14.8% and 37.9% at 25° C. and 40° C. storage temperatures, respectively. Without wishing to be bound by theory, the Molasses product when exposed to open air formed a dehydrated layer of crust that may be effective at preventing the escape of CHBr3 by creating a natural seal. This layer of crust did not form in the airtight samples, thus CHBr3 may escape gradually into the headspace of the vial and subsequently be released when the vials are opened for sampling.

The CHBr₃ content in Cyclodextrin is the most stable of the excipients tested with no significant changes at any of the temperatures and exposure conditions over the 4-week period. As of week 4, Cyclodextrin bound CHBr₃ does not appear to be negatively affected by higher temperatures (25° C. and 40° C.) or open-air conditions as observed with the Corn Oil or Molasses products. Small variations in CHBr₃ observed over time for all storage conditions (FIG. 11) may be attributed to analytical variation.

These results demonstrate that manufactured CHBr₃ can be stabilised and preserved in liquid (Corn Oil), semi-liquid (Molasses) and solid (Cyclodextrin) bromoform stabilising excipients, thereby providing stabilised bromoform compositions.

These results also demonstrate that the stabilised bromoform compositions described herein allow for appropriate concentrations of manufactured bromoform for effective methane (CH₄) mitigation in vitro and in vivo, and are suitable for ruminant diet formulation.

These results also demonstrate that the compositions described herein allow for sustained release of manufactured bromoform.

TABLE 2
Mean bromoform (CHBr3) content in each excipient over time (week) at different storage temperatures (Celsius) within airtight containers
and exposed to open air (25° C. and 40° C. only). Superscript letters indicate significantly different mean CHBr3 content
within excipients (P < 0.05). In treatments where there is a significant difference in CHBr3 over time the % change between week
0 and 4 has been expressed. Molasses open air samples were adjusted to account for percentage weight loss of each vial at each timepoint.

| | Storage conditions | Airtight containers | | | | | | | | Open air | | | |
|---|---|---|---|---|---|---|---|---|---|---|---|---|---|
| Sample | Temperature | −20° C. | | 4° C. | | 25° C. | | 40° C. | | 25° C. | | 40° C. | |
| Type | Time- week | mean | SE | mean | SE | mean | SE | mean | SE | mean | SE | mean | SE |
| Corn Oil | 0 | 3.02 ^{a} | 0.02 | 3.02 ^{a} | 0.02 | 3.02 ^{a} | 0.02 | 3.02 ^{a} | 0.02 | 3.02 ^{a} | 0.02 | 3.02 ^{a} | 0.02 |
| | 1 | 2.96 ^{a} | 0.02 | 3.02 ^{a} | 0.02 | 3.04 ^{a} | 0.01 | 3.03 ^{a} | 0.01 | 2.45 ^{b} | 0.10 | 2.37 ^{b} | 0.07 |
| | 2 | 2.94 ^{a} | 0.01 | 2.91 ^{a} | 0.01 | 2.93 ^{a} | 0.01 | 2.80 ^{b} | 0.05 | 2.46 ^{b} | 0.08 | 1.86 ^{c} | 0.13 |
| | 4 | 2.98 ^{a} | 0.01 | 2.96 ₐ | 0.01 | 2.95 ^{a} | 0.01 | 2.83 ^{b} | 0.02 | 1.78 ^{c} | 0.05 | 1.40 ^{d} | 0.07 |
| | % change over | — | | — | | — | | −6.3 | | −41.1 | | −53.6 | |
| Molasses | 4 weeks | 2.56 ^{a} | 0.02 | 2.56^{ab} | 0.02 | 2.56 ^{a} | 0.02 | 2.56 ^{a} | 0.02 | 2.56 ^{a} | 0.02 | 2.56 ^{a} | 0.02 |
| | 1 | 2.49 ^{a} | 0.04 | 2.63 ^{a} | 0.02 | 2.30 ^{b} | 0.05 | 1.85 ^{b} | 0.05 | 2.43^{ab} | 0.03 | 2.28 ^{b} | 0.01 |
| | 2 | 2.43 ^{a} | 0.02 | 2.49^{ab} | 0.03 | 2.02 ^{c} | 0.02 | 1.44 ^{c} | 0.12 | 2.29^{bc} | 0.04 | 1.86 ^{c} | 0.01 |
| | 4 | 2.45 ^{a} | 0.05 | 2.42 ^{b} | 0.04 | 1.50 ^{d} | 0.06 | 0.63 ^{d} | 0.07 | 2.18 ^{c} | 0.04 | 1.59 ^{d} | 0.02 |
| | % change over | — | | — | | −41.4 | | −75.4 | | −14.8 | | −37.9 | |
| Cyclo- | 4 weeks | 2.88 ^{a} | 0.08 | 2.88 ^{a} | 0.08 | 2.88 ^{a} | 0.08 | 2.88 ^{a} | 0.08 | 2.88 ^{a} | 0.08 | 2.88 ^{a} | 0.08 |
| dextrin | 1 | 2.88 ^{a} | 0.10 | 3.03 ^{a} | 0.06 | 2.99 ^{a} | 0.10 | 2.79 ^{a} | 0.05 | 2.94 ^{a} | 0.17 | 3.01 ^{a} | 0.09 |
| | 2 | 2.88 ^{a} | 0.06 | 3.01 ^{a} | 0.09 | 2.91 ^{a} | 0.04 | 2.87 ^{a} | 0.04 | 2.80 ^{a} | 0.08 | 2.79 ^{a} | 0.04 |
| | 4 | 2.85 ^{a} | 0.09 | 2.92 ^{a} | 0.09 | 3.16 a | 0.11 | 3.02 ^{a} | 0.07 | 2.98 ^{a} | 0.11 | 3.01 ^{a} | 0.07 |
| | % change over | — | | — | | — | | — | | — | | — | |
| | 4 weeks | | | | | | | | | | | | |

### Example 8: Asp-Oil Increases Growth Performance of Feedlot Cattle

On the basis of the results of Example 6 demonstrating Asp-Oil and a composition comprising manufactured bromoform and a stabilising excipient (canola oil) behave similarly in vivo, the present inventors sought to determine if the in vivo effects of the compositions described herein include changes in growth performance.

### Methods

The experimental design is a randomised block design including 30 pens of 10 animals each: 300 cattle total. All cattle were sourced from the same farm, similar breed (British cross) and sex (steers), with a target entry weight between 330-380 kg. The experimental design followed the Australian feedlot standard for retail meat of 80 days on feed to achieve the desired exit weight of 480-540 kg. A total of 3 ramp up diets were used to transition the steers from their pre-entry high forage diet to a high grain concentrate finisher ration, which is reflective of current feedlot practices. During these transitions, the respective total oil content of each ration increased in a stepwise procedure. The pre-starter ration is used only for the first few days after arrival to the feedlot. The starter, transition 1 (T1), and transition 2 (T2) diets were fed in 7 day blocks (21 days total), respectively.

Individual full body live weight (LW) measurements occurred upon arrival, at induction to the trial (day-5), start weight (day 0), mid weight (day 40), and end weight (day 80). ADWG was calculated for the full 80 days on feed (end weight-start weight/#days) and for the last 40 days to represent effect on the finisher diet (mid weight-end weight/40). The steers were fed for zero/low refusals based on previous day's intake. FCE was calculated for the full 80 days (DMI/ADWG) and for the last 40 days on feed (DMI days 40-80/ADWG days 40-80).

### Results

During the course of the experiment, the feedlot suffered significant weather events that resulted in flooded pens halfway through the trial. Twelve of the pens (out of 30) were relocated to a different area of the feedlot, which may have caused issues with feed intakes and weight gains therefore we are reporting the results in two forms [with and without flooded pens]. Table 2 reports data from all 30 pens whereas Table 3 reports data from the pens that were not flooded. This study demonstrated that compared to control steers (no Asp-Oil) the ADWG representing the full study duration (Days 1-81), improved by 2.2% (all pens) to 5.6% (flooded pens removed and FCE improved by 4.8% (all pens) to 5.2% (flooded pens removed). Feed Intake over days 1-81) had a different effect between all pens and with flooded pens removed with a decrease of 2.2% for all Asp-Oil pens compared to Control and an increase of 0.46% feed intake for non-flooded Asp-Oil pens compared to control. While this is an unusual effect, ADWG and FCE were still improved in Asp-Oil pens.

During the period between the final LW measurements the Asp-Oil was at its full feed inclusion level (Days 40-81; 34 mg CHBr3/kg DMI), and ADWG improved by 2.6% (all pens) to 6.6% (flooded pens removed), DMI decreased by 4.5% (all pens) to 1.24% (flooded pens removed), and FCE improved by 7.3% (all pens) to 8.0% (flooded pens removed). These results indicate that in animals fed with Asparagopsis biomass extracted into a bromoform stabilising excipient (Asp-Oil) there is an improvement of ADWG and FCE in beef cattle. Example 6 demonstrates that a composition comprising bromoform and the same bromoform stabilising excipient similarly reduces methane production in vivo; the present inventors propose the compositions comprising bromoform and a bromoform stabilising excipient improve growth performance on the same basis.

TABLE 3
Beef feedlot steers intake and growth parameters for
Control and Asparagopsis oil treatment groups. This
data table is inclusive of all 30 pens of animals.

| | units | Control | Asp Oil | Difference |
|---|---|---|---|---|
| Days 1-81 | | | | |
| Intake | kg as-fed/day | 14.67 | 14.33 | −2.3% |
| Average Daily | kg/day | 2.38 | 2.43 | 2.2% |
| Gain | | | | |
| Feed | ADG/Intake | 0.160 | 0.170 | 4.8% |
| Conversion | | | | |
| Efficiency | | | | |
| Days 40-81 | | | | |
| Intake | kg as-fed/day | 15.65 | 14.95 | −4.5% |
| Average Daily | Total Liveweight | 2.11 | 2.16 | 2.6% |
| Gain | Gain/# days | | | |
| Feed | ADG/Intake | 0.130 | 0.140 | 7.3% |
| Conversion | | | | |
| Efficiency | | | | |

TABLE 4
Beef feedlot steers intake and growth parameters for Control and
Asparagopsis oil treatment groups. This data table excludes
12 pens (out of 30) that flooded mid-way through the trial.

| | units | Control | Asp Oil | Difference |
|---|---|---|---|---|
| Days 1-81 | | | | |
| Intake | kg as-fed/day | 14.39 | 14.45 | 0.46% |
| Average Daily Gain | kg/day | 2.31 | 2.44 | 5.6% |
| Feed Conversion | ADG/Intake | 0.161 | 0.169 | 5.2% |
| Efficiency | | | | |
| Days 40-81 | | | | |
| Intake | kg as-fed/day | 15.33 | 15.14 | −1.24% |
| Average Daily Gain | Total Liveweight | 2.11 | 2.25 | 6.6% |
| | Gain/# days | | | |
| Feed Conversion | ADG/Intake | 0.138 | 0.149 | 8.0% |
| Efficiency | | | | |

1. A method for reducing total gas production and/or methane production in a ruminant animal comprising the step of providing said ruminant animal with an effective amount of a composition comprising manufactured bromoform and a bromoform stabilising excipient.

2. A method of claim 1 wherein the step of providing said ruminant animal an effective amount of a composition comprises administering said ruminant animal with the effective amount of a composition comprising manufactured bromoform in a bromoform stabilising excipient.

3. A method of claim 1 wherein the step of providing said ruminant animal an effective amount of a composition comprises making the composition comprising manufactured bromoform in a bromoform stabilising excipient available in a feed system comprising the ruminant animal.

4. A method of claim 2, wherein the composition is administered at a dose of at least 0.005, 0.01, 0.02, 0.03, 0.04, 0.05, 0.06 or 0.08 mg of bromoform per gram of organic matter provided to the ruminant animal.

5.-7. (canceled)

8. The method of any one of claim 1 wherein the bromoform stabilising excipient is an edible non-polar substance.

9. The method of claim 8 wherein the edible non-polar substance is an edible oil.

10. The method of claim 9 wherein the edible oil is selected from the group consisting of almond oil, apricot oil, argan oil, avocado oil, brazil nut oil, canola oil, cashew oil, coconut oil, colza oil, corn oil, copra oil, cottonseed oil, diacylglycerol oil, flaxseed oil, grapefruit seed oil, grapeseed oil, hazelnut oil, hemp oil, lemon oil, linseed oil, macadamia oil, mustard oil, olive oil, orange oil, palm oil, palm kernel oil, peanut oil, pecan oil, pine nut oil, pistachio oil, pumpkin seed oil, rapeseed oil, rice bran oil, safflower oil, sesame oil, soybean oil, sunflower oil, walnut oil, and vegetable oil, or any combination thereof.

11. The method of any one of claim 1 wherein the bromoform stabilising excipient is an edible carbohydrate, or water.

12. The method of claim 11 wherein the carbohydrate is a cyclodextrin or a molasses.

13. A method according to any one of claim 1 wherein the composition is in a solid, a semi-solid or a liquid form.

14.-44. (canceled)

45. A stabilised bromoform composition comprising manufactured bromoform and a bromoform stabilising excipient, wherein the bromoform stabilising excipient is selected from the group consisting of an edible solid, edible semi solid or edible liquid.

46. A stabilised bromoform composition according to claim 45, wherein the bromoform stabilising excipient is selected from the group consisting of an edible oil, an edible carbohydrate and water.

47. A stabilised bromoform composition according to claim 45, wherein the composition does not comprise one or more compounds selected from the group consisting of iodine, bromine, dibromochloromethane, bromochloroacetic acid, and dibromoacetic acid.

48. A method of making a stabilised bromoform composition comprising contacting manufactured bromoform and a bromoform stabilising excipient, wherein the bromoform stabilising excipient is selected from the group consisting of an edible solid, edible semi solid or edible liquid.

49. A method of making a stabilised bromoform composition according to claim 48, wherein the bromoform stabilising excipient is selected from the group consisting of an edible oil, an edible carbohydrate and water.

50. A method according to claim 48, wherein the composition does not comprise one or more compounds selected from the group consisting of iodine, bromine, dibromochloromethane, bromochloroacetic acid, and dibromoacetic acid.
